(19) United States
(12) Patent Application Publication
(10) Pub. No.: US 2026/0107972 A1
(43) Pub. Date: Apr. 23, 2026

(54) AEROSOL GENERATING GEL MATERIAL COMPRISING A SUBSTITUTED 3-(1 METHYLPYRROLIDIN-2-YL)PYRIDINE

(71) Applicant: RAI STRATEGIC HOLDINGS, INC., Winston-Salem, NC (US)

(72) Inventors: Kyle Ford, Pfafftown, NC (US); Brandon S. Darrow, Winston-Salem, NC (US); Michael R. Galloway, Winston-Salem, NC (US); Timothy O. Williams, Walnut Cove, NC (US); Gary M. Dull, Lewisville, NC (US)

(21) Appl. No.: 19/361,724

(22) Filed: Oct. 17, 2025

Related U.S. Application Data
(60) Provisional application No. 63/708,875, filed on Oct. 18, 2024.
(60) Provisional application No. 63/880,556, filed on Sep. 12, 2025.

(51) Int. Cl. A24B 15/167; A24B 15/30; A24D 1/00; A24D 1/02; A24D 1/18; A24D 1/20; A24F 40/20; A24F 40/46; A24F 40/465; A61K 9/00; A61K 31/00; A61K 31/4045; A61K 31/4427; A61K 31/465; A61K 31/522

(52) U.S. Cl. CPC A24B 15/167; A24B 15/303; A24D 1/002; A24D 1/02; A24D 1/18; A24D 1/20; A24F 40/20; A24F 40/46; A24F 40/465; A61K 9/007; A61K 31/4045; A61K 31/4427; A61K 31/465; A61K 31/522; A61K 31/658

(57) ABSTRACT

Aerosol generating compositions adapted for use in an aerosol delivery device are provided. The aerosol generating compositions include a thin film. The thin film includes a binder, an aerosol former, and an active agent, wherein the active agent includes at least a substituted 3-(1-methylpyrrolidin-2-yl)pyridine, 3-(azetidin-2-yl)pyridine, or 3-(azetidin-2-ylmethoxy)pyridine. The disclosure further provides articles and devices incorporating such aerosol generating compositions.

### CROSS-REFERENCE TO RELATED APPLICATIONS

This application claims priority to U.S. Provisional Application No. 63/708,875, filed on Oct. 18, 2024, and claims priority to U.S. Provisional Application No. 63/880,556, filed on Sep. 12, 2025, each of which is incorporated herein by reference in its entirety and for all purposes.

### FIELD OF THE DISCLOSURE

The present disclosure relates to aerosol generating materials and articles for use in an aerosol provision system, to aerosol provision systems including such materials and articles, and to methods for forming the aerosol generating materials and articles.

### BACKGROUND

Smoking articles such as cigarettes, cigars and the like burn tobacco during use to create tobacco smoke. Alternatives to these types of combustible articles release an inhalable aerosol or vapor by releasing compounds from a substrate material by heating without burning. These may be referred to as non-combustible smoking articles or aerosol generating articles.

One example of such a product is a heating device which release compounds by heating, but not burning, a solid aerosolizable material. This solid aerosolizable material may, in some embodiments, contain a tobacco material. The heating volatilizes at least one component of the material, typically forming an inhalable aerosol. These products may be referred to as heat-not-burn devices, tobacco heating devices or tobacco heating products. Various different arrangements for volatilizing at least one component of the solid aerosolizable material are known.

### BRIEF SUMMARY

The present disclosure is generally directed to an aerosol generating composition comprising a thin film. The thin film comprises a binder, an aerosol former, and an active agent, the active agent comprising at least a substituted 3-(1-methylpyrrolidin-2-yl)pyridine, a 3-(azetidin-2-yl)pyridine, or a 3-(azetidin-2-ylmethoxy)pyridine.

The present disclosure includes, without limitation, the following embodiments.

Embodiment 1: An aerosol generating composition comprising a thin film, the thin film comprising a binder, an aerosol former, and an active agent, wherein the active agent comprises at least at least a compound having a structure according to Formula I, Formula II, or Formula III:

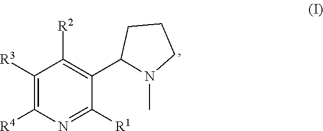

wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or

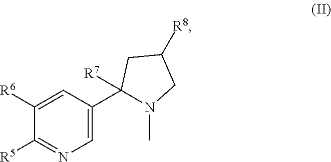

wherein:
R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C₁-C₃ alkyl; and
at least one of R⁷ and R⁸ is not hydrogen; or

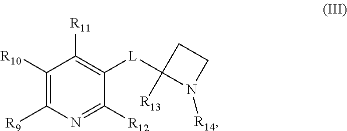

wherein:
L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

Embodiment 2: The aerosol generating composition of embodiment 1, wherein the active agent comprises a compound having a structure according to Formula I, and wherein R¹, R², and R³ are each H.

Embodiment 3: The aerosol generating composition of embodiment 1 or 2, wherein the active agent comprises a compound having a structure according to Formula I, and wherein R⁴ is optionally substituted C₁-C₆ alkyl, F, Cl, Br, OCH₃, OEt, or CN.

Embodiment 4: The aerosol generating composition of embodiment 3, wherein R⁴ is C₁-C₃ alkyl, such as wherein R⁴ is CH₃.

Embodiment 5: The aerosol generating composition of embodiment 1, wherein the active agent comprises a compound having a structure according to Formula II, wherein R⁵ and R⁶ are H; R⁷ is CH₃; and R⁸ is H.

Embodiment 6: The aerosol generating composition of embodiment 1, wherein the active agent comprises a compound having a structure according to Formula I, wherein R⁵, R⁶ and R⁷ are H, and R⁸ is CH₃.

Embodiment 7: The aerosol generating composition of any one of embodiments 1-6, wherein the thin film further comprises a botanical extract, a tobacco extract, or a combination thereof.

Embodiment 8: The aerosol generating composition of any one of embodiments 1-7, further comprising a botanical material.

Embodiment 9: The aerosol generating composition of embodiment 8, wherein the botanical material comprises tobacco.

Embodiment 10: The aerosol generating composition of embodiment 8, wherein the botanical material comprises a botanical extract.

Embodiment 11: The aerosol generating composition of embodiment 8, wherein the botanical material comprises a shredded non-tobacco botanical material; optionally, wherein the shredded non-tobacco botanical material comprises or is rooibos.

Embodiment 12: The aerosol generating composition of embodiment 8, wherein the aerosol generating composition comprises from about 50 to about 95% of the botanical material and from about 5 wt % to about 50 wt % of the thin film.

Embodiment 13: The aerosol generating composition of embodiment 8, wherein the thin film is present as a cut or shredded sheet which is blended with the botanical material.

Embodiment 14: The aerosol generating composition of embodiment 8, comprising a plug or section comprising the botanical material, wherein the thin film is present in the form of a sheet circumscribing at least a portion of the plug or section.

Embodiment 15: The aerosol generating composition of embodiment 8, further comprising a paper wrapper circumscribing the aerosol generating composition, and wherein the thin film is present in the form of a sheet positioned between the botanical material and the wrapper.

Embodiment 16: The aerosol generating composition of embodiment 8, comprising a first section comprising the botanical material and a second section comprising the thin film in the form of a rolled sheet.

Embodiment 17: The aerosol generating composition of any one of embodiments 1-16, wherein the active agent further comprises a nicotine component, a cannabinoid, a terpene, caffeine, an amino acid, a vitamin, melatonin, a botanical extract, or a combination thereof.

Embodiment 18: The aerosol generating composition of any one of embodiments 1-16, wherein the active agent further comprises a nicotine component.

Embodiment 19: The aerosol generating composition of any one of embodiments 1-16, wherein the aerosol generating composition is substantially free of a nicotine component.

Embodiment 20: The aerosol generating composition of any one of embodiments 1-19, further comprising a second thin film, the second thin film comprising a binder, an aerosol former, and optionally, an active agent.

Embodiment 21: An aerosol generating article configured for use in an aerosol provision system, the aerosol generating article comprising the aerosol generating composition of any one of embodiments 1-20.

Embodiment 22: An aerosol provision system for heating an aerosol generating composition to volatilize at least one component of the aerosol generating composition, the system comprising:
the aerosol generating article of embodiment 21; and
a heating device configured to receive at least a portion of the article and to heat the aerosol generating composition to volatilize at least one component of the aerosol generating composition.

Embodiment 23: The aerosol provision system of embodiment 22, wherein the heating device is configured to externally heat the aerosol generating composition, inductively heat the aerosol generating composition, resistively heat the aerosol generating composition, or a combination thereof.

Embodiment 24: The aerosol generating composition of embodiment 1, wherein the active agent has a structure according to Formula II, optionally wherein R⁵ is optionally substituted C₁-C₆ alkyl, F, Cl, Br, OCH₃, OCH₂CH₃, or CN; and R⁶ is H, or optionally wherein R⁵ is H; and R⁶ is optionally substituted C₁-C₆ alkyl, F, Cl, Br, OCH₃, OCH₂CH₃, or CN.

Embodiment 25: The article of embodiment 1, wherein the active agent has a structure according to Formula III, optionally wherein R⁹ is H or CH₃; and R¹⁰, R¹¹, and R¹² are each H.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise.

### BRIEF DESCRIPTION OF THE DRAWINGS

Having thus described aspects of the disclosure in the foregoing general terms, reference will now be made to the accompanying figures, which are not necessarily drawn to scale, and wherein:

FIG. 1 is a side-on cross-sectional view of an article for use in an aerosol provision system according to a non-limiting embodiment of the disclosure.

FIG. 2 is a side-on cross-sectional view of an article according to a non-limiting embodiment of the disclosure.

FIG. 3 is a side-on cross-sectional view of an article according to a non-limiting embodiment of the disclosure.

FIG. 4 is a side-on cross-sectional view of an article according to a non-limiting embodiment of the disclosure.

FIG. 5 is a perspective illustration of a non-combustible aerosol provision device for generating aerosol from an article according to a non-limiting embodiment of the disclosure.

FIG. 6 is a graphical depiction showing the transfer efficiency of 2-methyl-5-(1-methylpyrrolidin-2-yl)pyridine from an aerosol generating rod to aerosol as compared to a that of a control aerosol generating rod including nicotine.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

As used in this specification and the claims, the singular forms “a,” “an,” and “the” include plural referents unless the context clearly dictates otherwise.

Reference to “dry weight percent” or “dry weight basis” refers to weight on the basis of dry ingredients (i.e., all ingredients except water). All weight percent values herein are dry weight percent unless otherwise indicated.

The terms ‘upstream’ and ‘downstream’ used herein are relative terms defined in relation to the direction of mainstream aerosol drawn though an article or device in use.

Unless otherwise defined herein, by “substantially free” it is meant that the noted component (e.g., nicotine, acid, or a botanical material) has not been intentionally added, beyond trace amounts that may be present e.g., as an impurity in another component, or small amounts which may be present in certain flavor packages. For example, some embodiments can have less than 0.01% by weight of the noted component, or less than 0.001%, or even 0% by weight of the noted component, based on the total weight of the aerosol generating material. In some embodiments, the aerosol generating material is completely free of the noted component (i.e., having 0% or as having an amount below the limit of detection).

The present disclosure is generally directed to an aerosol generating composition comprising a thin film, the thin film comprising a binder, an aerosol former, and an active agent, wherein the active agent comprises at least a substituted 3-(1-methylpyrrolidin-2-yl)pyridine, a 3-(azetidin-2-yl)pyridine, or a 3-(azetidin-2-ylmethoxy)pyridine. Each of the articles, devices, materials, and components of each thereof are further described herein below.

### Aerosol Generating Composition

The present disclosure provides aerosol generating compositions and articles and systems comprising such compositions. A significant challenge associated with aerosol provision systems is the phenomenon known as “hot puff”. This is reference to the steam that is given off when the aerosol generating material is first heated. As the temperature rises, water evaporates, forming steam which the user can inhale. The greater the water content of the aerosol generating material, the greater the amount of steam generated on heating and the increased hot puff. This is unpleasant and various approaches have been proposed to reduce or prevent hot puff.

Ventilation can be an important feature of an aerosol generating article for reducing the hot puff phenomenon. Ventilation allows ambient air to be drawn into the aerosol generating article, which mixes with the aerosol generated by heating the aerosol generating material. The ambient air cools the aerosol and may cause condensation of some of the gaseous elements, thus reducing hot puff. However, another effect of ventilation is that it dilutes the aerosol, and therefore reduces the perceived impact of the aerosol to the user, affecting delivery of the active and/or flavor. Countering the hot puff with ventilation will, however, reduce the satisfaction that the (diluted) aerosol provides the user.

The present disclosure provides aerosol generating compositions and articles comprising such compositions which address the foregoing issues. Accordingly, in one aspect is provided an aerosol generating composition comprising a thin film, the thin film comprising a binder, an aerosol former, and an active agent, wherein the active agent comprises at least a substituted 3-(1-methylpyrrolidin-2-yl)pyridine, a 3-(azetidin-2-yl)pyridine, or a 3-(azetidin-2-ylmethoxy)pyridine.

The aerosol generating compositions comprising such thin films generally have a moisture content of no greater than about 15% by weight. The articles comprising such compositions include ventilation and, specifically have no more than about 50% ventilation based on the volume of aerosol generated by the aerosol generating composition passing through the article when the article is heated by an aerosol delivery device. The combination of the relatively low water content of the aerosol generating composition and the relatively low level of ventilation means that the aerosol generating articles provide an impactful aerosol without the risk of the user experiencing hot puff.

Further, the thin film provides a number of beneficial properties to the aerosol generating composition. For example, the components provided by the thin film (e.g. active agent(s), flavors or both) provide a concentrated source of such substances. This means that the amount of such thin film included in the aerosol generating composition may be reduced compared to the amounts of thin films that are included in conventional aerosol generating compositions. For example, the aerosol generating composition may include as little as about 5 wt % of the thin film, whilst still contributing to the generation of a flavorful and impactful aerosol. The thin film also provides these volatile components in a stabilized form. The thin film also provides an aerosol generating composition with a controlled water content, e.g., remaining at or below about 15 wt %. The thin film may also represent a source of aerosol former(s) that can be difficult to incorporate into an aerosol generating composition in large enough amounts to provide the desired aerosol body and visible aerosol.

Thus, the aerosol generating articles according to the present disclosure provide the user with an inhalable medium with desirable sensory properties as a result of the concentrated form of the components provided by the thin film, and the reduced ventilation that is required as a result of the low moisture content of the aerosol generating composition.

A reduction in the amount of the thin film present in the aerosol generating composition may also afford additional flexibility in terms of how this material is incorporated in the aerosol generating composition and in the design of articles incorporating the aerosol generating composition. This allows articles to be designed that permit controlled heating of the thin film and controlled generation of an aerosol from the aerosol generating composition.

The inclusion of a relatively small amount of thin film may also mean a reduction in the total mass of the aerosol generating composition and of the aerosol generating article, with consequent lower costs and potentially lower energy requirements.

There may also be a further benefit in that the sensory quality of the initial puffs, such as the first 1 or 2 puffs, may be improved, leading to a better user experience and increased user satisfaction.

### Thin Film

The aerosol generating composition as disclosed herein comprises a thin film. As referred to herein, the thin film is a sheet material that may be heated to form an aerosol. The thin film may alternatively be referred to as a “dried gel”. The thin film is a solid material that may retain some fluid, such as liquid, within it. In some embodiments, the thin film is optionally cast or extruded to form the sheet. The thin film comprises a binder, such as a gelling agent, an aerosol former, and an active agent. Optionally, components including but not limited to flavorants, fillers, and solvents such as water may also be present.

The thin film may have a thickness of from about 0.015 mm to about 1 mm. For example, the thickness may be in the range of from about 0.05 mm, about 0.1 mm or about 0.15 mm to about 0.5 mm or about 0.3 mm.

The thin film may comprise more than one layer or film, and the thickness described herein may refer to the aggregate thickness of those layers or films.

The thin film may be continuous. For example, the film may comprise or be a continuous sheet of material. The sheet may be in the form of a wrapper, it may be gathered to form a gathered sheet, or it may be shredded to form a shredded sheet. The shredded sheet may comprise one or more strands or strips of thin film.

The thin film may be a “monolithic solid”. The thin film may be substantially non-fibrous. In some embodiments, the thin film may be a dried gel. The thin film may retain some fluid, such as liquid, within it.

### Binder

The thin film as disclosed herein comprises a binder that binds the components together to form a cohesive whole. The binder may be a gelling agent.

In some embodiments, the thin film may comprise from about 1 wt %, about 2 wt %, about 3 wt %, about 4 wt %, about 5 wt %, about 10 wt %, about 15 wt % or about 20 wt %, to about 60 wt %, about 50 wt %, about 40 wt %, about 30 wt %, about 25 wt %, about 20 wt %, about 15 wt %, or about 10 wt % of one or more binders on a dry-weight basis (DWB). For example, the thin film may comprise from about 1 to about 40 wt %, from about 2 to about 20 wt % or from about 4 to about 15 wt % of binder (DWB). Suitably, the thin film may comprise from about 1 wt % to about 60 wt % gelling agent, for example from about 1 wt %, about 2 wt %, about 3 wt %, about 4 wt %, about 5 wt %, about 10 wt %, about 15 wt % or about 20 wt %, to about 60 wt %, about 50 wt %, about 40 wt %, about 30 wt %, about 25 wt %, about 20 wt %, about 15 wt %, about 10 wt % of a gelling agent (all calculated on a dry weight basis). For example, the thin film may comprise from about 1 to about 30 wt %, from about 2 to about 20 wt % or from about 4 to about 15 wt % of the gelling agent.

In some embodiments, the gelling agent comprises a hydrocolloid. In some embodiments, the gelling agent comprises (or is) one or more compounds selected from polysaccharide gelling agents, such as alginate, pectin, starch or a derivative thereof, cellulose or a derivative thereof, pullulan, carrageenan, agar and agarose; gelatin; gums, such as xanthan gum, guar gum and acacia gum; silica or silicone compounds, such as PDMS and sodium silicate; clays, such as kaolin; and polyvinyl alcohol.

In some embodiments the gelling agent comprises (or is) one or more polysaccharide gelling agents. In some embodiments, the polysaccharide gelling agent is selected from alginate, pectin, starch or a derivative thereof, or cellulose or a derivative thereof. In some embodiments the polysaccharide gelling agent is selected from alginate and a cellulose derivative. In some embodiments, the gelling agent is a polysaccharide gelling agent, optionally wherein the polysaccharide gelling agent is selected from alginate and a cellulose derivative. In some embodiments, the alginate is sodium alginate. In some embodiments the gelling agent is not crosslinked. Without wishing to be bound by theory, it is believed that the absence of crosslinks in the gelling agent facilitates quicker delivery of the active substances and/or flavors from the thin film material.

In some embodiments, the polysaccharide gelling agent is a cellulose derivative, also referred to herein as a cellulosic gelling agent. Examples of cellulose derivatives include, but are not limited to, hydroxymethyl cellulose, hydroxyethyl cellulose, hydroxypropyl cellulose, carboxymethylcellulose (CMC), hydroxypropyl methylcellulose (HPMC), methyl cellulose, ethyl cellulose, cellulose acetate (CA), cellulose acetate butyrate (CAB), and cellulose acetate propionate (CAP). In some embodiments the cellulose or derivative thereof is selected from hydroxymethyl cellulose, hydroxyethyl cellulose, hydroxypropyl cellulose, carboxymethylcellulose (CMC), hydroxypropyl methylcellulose (HPMC), methyl cellulose, ethyl cellulose, cellulose acetate (CA), cellulose acetate butyrate (CAB), and cellulose acetate propionate (CAP). In some embodiments, the cellulose derivative is CMC.

In some embodiments, the gelling agent comprises (or is) one or more of alginate, pectin, hydroxyethyl cellulose, hydroxypropyl cellulose, carboxymethylcellulose, pullulan, xanthan gum guar gum, carrageenan, agarose, acacia gum, fumed silica, PDMS, sodium silicate, kaolin and polyvinyl alcohol.

In some embodiments, the gelling agent comprises (or is) one or more of hydroxyethyl cellulose, hydroxypropyl cellulose, carboxymethylcellulose, guar gum, acacia gum, alginate and/or pectin.

In some embodiments, the gelling agent comprises (or is) alginate and/or pectin and may be combined with a setting agent (such as a calcium source) during formation of the thin film. In some embodiments, the aerosol-generating material may comprise a calcium-crosslinked alginate and/or a calcium-crosslinked pectin.

In some embodiments, the gelling agent comprises (or is) alginate, optionally wherein the alginate is present in the aerosol generating composition in an amount of from about 1 to about 10 wt %, for example from about 3 to about 6 wt %, of the aerosol generating composition (DWB).

In some embodiments, alginate is the only gelling agent present in the thin film. In other embodiments, the gelling agent comprises alginate and at least one further gelling agent, such as pectin.

In some embodiments, the gelling agent is carboxymethylcellulose, optionally wherein the carboxymethylcellulose (CMC) is present in an amount of from about 15 to about 50 wt %, for example from about 20 to about 40 wt % or about 30 wt %. In some embodiments, CMC is the only gelling agent present in the thin film.

### Filler

In some embodiments, the thin film comprises a filler. The filler may be present to adjust the physical and/or chemical properties of the film. For example, in some embodiments, the filler may increase the tensile strength of the thin film and render it more suitable for large scale manufacture of aerosol generating articles. On the other hand, inclusion of a filler may add to the cost, weight and density of the thin film. Adding to the mass of the thin film potentially adds to the energy and time required to heat the film to generate the desired aerosol.

In some embodiments, the thin film comprises no more than about 60 wt % of a filler, such as from about 1 wt % to about 60 wt %, or from about 5 wt % to about 50 wt %, or from about 5 wt % to about 30 wt %, or from about 10 wt % to about 20 wt %.

In some embodiments, the thin film comprises no more than about 20 wt %, suitably no more than about 10 wt % or no more than about 5 wt % of a filler. In some embodiments, the thin film comprises no more than about 1 wt % of a filler, and in some embodiments, comprises no filler.

The filler, when present, may comprise one or more inorganic filler materials, such as calcium carbonate, perlite, vermiculite, diatomaceous earth, colloidal silica, magnesium oxide, magnesium sulphate, magnesium carbonate, and suitable inorganic sorbents, such as molecular sieves. The filler may comprise one or more organic filler materials such as wood pulp; tobacco pulp; hemp fiber; starch and starch derivatives, such as maltodextrin; chitosan; and cellulose and cellulose derivatives, such as microcrystalline cellulose and nanocrystalline cellulose. In particular embodiments, the thin film comprises no calcium carbonate such as chalk.

In particular embodiments which include filler, the filler is fibrous. For example, the filler may be a fibrous organic filler material such as wood pulp, hemp fiber, cellulose or cellulose derivatives. In some embodiments, the fibrous filler is wood pulp. Without wishing to be bound by theory, it is believed that including fibrous filler in a thin film may increase the tensile strength of the material. This may be particularly advantageous in examples wherein the thin film is provided as a sheet, such as when a thin film sheet circumscribes a rod of aerosolizable material.

In some embodiments, the thin film does not comprise tobacco fibers. In some embodiments, the thin film does not comprise fibrous material.

In some embodiments the gelling agent is cellulose, such as CMC, and/or guar gum, and is used together with wood pulp as a filler.

The thin film may, in some embodiments, comprise water. In some instances, the thin film is a hydrogel. In some embodiments, the thin film comprises no more than about 20 wt %, about 15 wt %, about 12 wt %, about 10 wt %, about 9 wt % or about 8 wt % water. In some embodiments, the thin film may comprise at least about 1 wt %, about 2 wt % or about 5 wt % water. The thin film may comprise about 10 wt % water. In some embodiments, the thin film comprises from about 5 wt % to about 10 wt % water, or from about 5 wt % to about 9 wt %. Suitably, the water content of the thin film may be about 5 wt %.

### Aerosol Former

The thin film of the aerosol generating composition as disclosed herein comprises an aerosol former. In this context, an “aerosol former” or “aerosol forming material” is an agent that promotes the generation of an aerosol. An aerosol former may promote the generation of an aerosol by promoting an initial vaporization and/or the condensation of a gas to an inhalable solid and/or liquid aerosol. In some embodiments, an aerosol former may improve the delivery of active agent, flavor, or both from the aerosol generating composition.

The thin film may comprise from about 5 wt %, about 10 wt %, about 15 wt %, about 20 wt %, about 25 wt %, about 27 wt % or about 30 wt % to about 60 wt %, about 55 wt %, about 50 wt %, about 45 wt %, about 40 wt %, or about 35 wt % of an aerosol former (DWB). For example, the thin film may comprise from about 10 to about 60 wt %, from about 20 to about 50 wt %, from about 25 to about 40 wt % or from about 30 to about 35 wt % of an aerosol former.

Suitably, the thin film may comprise from about 0.1 wt %, about 0.5 wt %, about 1 wt %, about 3 wt %, about 5 wt %, about 7 wt % or about 10 wt % to about 50 wt %, about 45 wt %, about 40 wt %, about 35 wt %, about 30 wt % or about 25 wt % of an aerosol former (DWB). For example, the thin film may comprise from about 0.5 to about 40 wt %, from about 3 to about 35 wt % or from about 10 to about 25 wt % of an aerosol former.

In general, any suitable aerosol former may be included in the thin film of the disclosure. The particular choice of aerosol former(s) may depend on factors such as the method of aerosol formation, the appearance and volume of the aerosol, the desired density of the aerosol, and the like. Suitable aerosol formers include, but are not limited to: a polyol such as sorbitol, glycerol, and glycols like propylene glycol or triethylene glycol; a non-polyol such as monohydric alcohols, high boiling point hydrocarbons, acids such as lactic acid, glycerol derivatives, esters such as diacetin, triacetin, triethylene glycol diacetate, triethyl citrate or myristates including ethyl myristate and isopropyl myristate and aliphatic carboxylic acid esters such as methyl stearate, dimethyl dodecanedioate and dimethyl tetradecanedioate. In some embodiments, the aerosol former may be glycerol, propylene glycol, or a mixture of glycerol and propylene glycol.

In some embodiments, the aerosol former may comprise one or more of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

In some embodiments, the aerosol former comprises one or more compound selected from erythritol, propylene glycol, glycerol, triacetin, sorbitol and xylitol. In some embodiments, the aerosol-former material comprises, consists essentially of, or consists of, glycerol.

In some embodiments, the aerosol former comprises a mixture of glycerol and propylene glycol in a weight ratio of glycerol to propylene glycol of from about 3:1 to about 1:3, from about 2:1 to about 1:2, from about 1.5:1 to about 1:1.5, from about 55:45 to about 45:55, or about 45:55.

The aerosol former may act as a plasticizer for the thin film. It has been established that if the content of the aerosol former is too high, the thin film may absorb water (as the aerosol former is hygroscopic) resulting in a material that does not create an appropriate consumption experience in use. It has also been established that if the aerosol former content is too low, the thin film may be brittle and easily broken (as the aerosol former may act as a plasticizer). The aerosol former content specified herein provides a thin film with flexibility which allows the thin film to be wound onto a bobbin, which is useful in manufacture of aerosol generating articles.

### Active Agent

The thin film of the aerosol generating composition of the disclosure comprises an active agent, including, but not limited to, a substituted 3-(1-methylpyrrolidin-2-yl)pyridine. The active agent may further include components such as botanical materials, cannabinoids, terpenes, vitamins, melatonin, caffeine, tobacco extract, or combinations thereof. The various components are described further herein below.

As used herein, the term “substituted 3-(1-methylpyrrolidin-2-yl)pyridine” refers to a compound having a 3-(1-pyrrolidin-2-yl)pyridine) scaffold and bearing one or more non-hydrogen substituents on the pyridine ring.

In some embodiments, the substituted 3-(1-methylpyrrolidin-2-yl)pyridine has a structure according to Formula I:

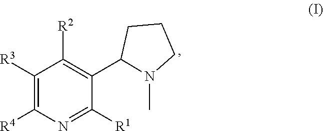

wherein:
R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and at least one of R¹, R², R³, and R⁴ are not hydrogen.

Substituted 3-(1-methylpyrrolidin-2-yl)pyridines with various R¹, R², R³, and R⁴ substituents have been reported previously. See for example, U.S. Pat. Nos. 4,321,387, 4,155,909; 5,015,741, 5,138,062, and 5,703,100, each of which is incorporated by reference herein and describe such substituted 3-(1-methylpyrrolidin-2-yl)pyridines, their synthesis, and pharmacological properties. Substituted 3-(1-methylpyrrolidin-2-yl)pyridines and their pharmacological profiles have also been disclosed in Wang et al., Drug Development Research 1998, Volume 45, Issue 1, Pages 10-16; and Dukat et al. European Journal of Medicinal Chemistry 1999, 34(1): 31-40.

In some embodiments, R¹, R², and R⁴ are each H, and R³ is a non-hydrogen substituent. In some embodiments, R³ is optionally substituted C₁-C₆ alkyl, F, Cl, Br, OMe, OEt, or CN. In some embodiments, R³ is Me or Et. In some embodiments, R³ is F.

In some embodiments, R¹, R², and R³ are each H, and R⁴ is a non-hydrogen substituent.

In some embodiments, R⁴ is optionally substituted C₁-C₆ alkyl, F, Cl, Br, OMe, OEt, or CN.

In some embodiments, R¹, R², and R³ are each H, and R⁴ is optionally substituted C₁-C₆ alkyl, F, Cl, Br, OCH₃, OEt, or CN.

In some embodiments, R¹, R², and R³ are each H, and R⁴ is C₁-C₃ alkyl.

In some embodiments, R¹, R², and R³ are each H, and R⁴ is Me. In such embodiments, the compound of Formula I may be referred to as 2-methyl-5-(1-methylpyrrolidin-2-yl)pyridine.

The pharmacology of 2-methyl-5-(1-methylpyrrolidin-2-yl)pyridine has been reported in, for example, Dukat et al. European Journal of Medicinal Chemistry, Volume 31, Issue 11, 1996, Pages 875-888 (incorporated herein by reference), and the pharmacological profile of the (S)-enantiomer of 2-methyl-5-(1-methylpyrrolidin-2-yl)pyridine in the form of the benzoate salt (CAS 2861225-70-7; referred to as Imotine™) is discussed in Carmines et al, Poster #6; 76^{th} TSRC Conference 2023, Norfolk, VA, USA).

In some embodiments, the substituted 3-(1-methylpyrrolidin-2-yl)pyridine has a structure according to Formula II:

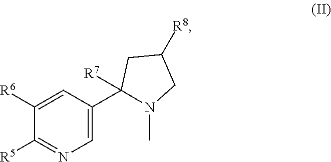

wherein:
R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C₁-C₃ alkyl; and
at least one of R⁷ and R⁸ is not hydrogen.

Certain substituted 3-(1-methylpyrrolidin-2-yl)pyridines with various R⁵, R⁶, R⁷, and R⁸ substituents have been reported previously. See for example, U.S. Pat. Nos. 4,321,387, 4,155,909; 5,015,741, 5,138,062, and 5,703,100, each of which is incorporated by reference herein and describe example substituted 3-(1-methylpyrrolidin-2-yl)pyridines, their synthesis, and pharmacological properties. Certain substituted 3-(1-methylpyrrolidin-2-yl)pyridines and their pharmacological profiles have also been disclosed in Wang et al., Drug Development Research 1998, Volume 45, Issue 1, Pages 10-16; Dukat et al. European Journal of Medicinal Chemistry 1999, 34(1): 31-40; Lin et al., J. Med. Chem. (1994), 37, 3542-3553.

In some embodiments, R⁵ and R⁶ are H; R⁷ is CH₃; and R⁸ is H. In such embodiments, the compound of Formula II may be referred to as 3-(1,2-dimethylpyrrolidin-2-yl)pyridine, or 2′-methyl-5-(1-methylpyrrolidin-2-yl)pyridine, and has a structure:

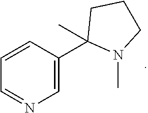

The compound 3-(1,2-dimethylpyrrolidin-2-yl)pyridine is known in the literature, and has a Chemical Abstracts Registry Number of 220650-38-4. The synthesis of this compound has been reported in Rouchaud et al., J Het Chem 2012, 49(1), 161-166; Wang et al., Drug Dev Res 1998, 45(1), 10-16; Secor et al., Tetrahedron Lett. (1981), 22(33), 3151-3154; US Patent Application Publication No. 2013/0157995; PCT Application Publication No. WO2012/031220; and U.S. Pat. No. 9,440,948, each of which are incorporated by reference herein with respect to the synthesis of 3-(1,2-dimethylpyrrolidin-2-yl)pyridine.

In some embodiments, R⁵ and R⁶ are H; R⁷ is H; and R⁸ is CH₃. In such embodiments, the compound of Formula II may be referred to as 3-(1,4-dimethylpyrrolidin-2-yl)pyridine, or 4′-methyl-5-(1-methylpyrrolidin-2-yl)pyridine, and has a structure:

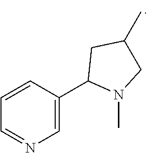

The compound 3-(1,4-dimethylpyrrolidin-2-yl)pyridine is known in the literature, has a Chemical Abstracts Registry Number of 74805-00-8, and is commercially available from, for example, Enamine Stock Building Blocks and Aurora Building Blocks. The synthesis of this compound has been reported in U.S. Pat. No. 9,440,948; and EP Patent No. 559495, each of which are incorporated by reference herein with respect to the synthesis of 3-(1,4-dimethylpyrrolidin-2-yl)pyridine.

In some embodiments, R⁵ is CH₃; R⁶ is H; R⁷ is CH₃; and R⁸ is H. In such embodiments, the compound of Formula II may be referred to as 5-(1,2-dimethylpyrrolidin-2-yl)-2-methylpyridine, and has a structure:

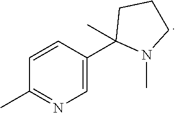

The compound 5-(1,2-dimethylpyrrolidin-2-yl)-2-methylpyridine may be readily synthesized according to known reactions. For example, commercially available 2-methyl-5-(2-methylpyrrolidin-2-yl)pyridine (Chemical Abstracts Registry Number of 1528955-30-7; Aurora Building Blocks, Adlab Chemicals Building Blocks) can be N-methylated with formaldehyde and formic acid, or alternatively with formaldehyde and a reducing agent such as sodium cyanoborohydride to afford 5-(1,2-dimethylpyrrolidin-2-yl)-2-methylpyridine. Alternatively, 5-(1,2-dimethylpyrrolidin-2-yl)-2-methylpyridine may be synthesized by lithiation of 2-methyl-5-bromopyridine, reaction of the lithiated pyridine with N-methylpyrrolidone, and addition of methyl lithium to the perchlorate salt of the resulting imine. This reaction sequence is shown below in Scheme 1.

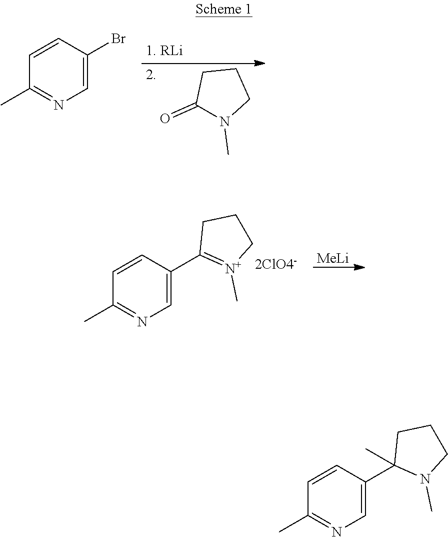

In some embodiments, R⁵ is H or CH₃, R⁶ is H, R⁷ is H or CH₃, and R⁸ is H or CH₃, provided that at least one of R⁷ and R⁸ is CH₃. Accordingly, in some embodiments, the substituted 3-(1-methylpyrrolidin-2-yl)pyridine has a structure selected from

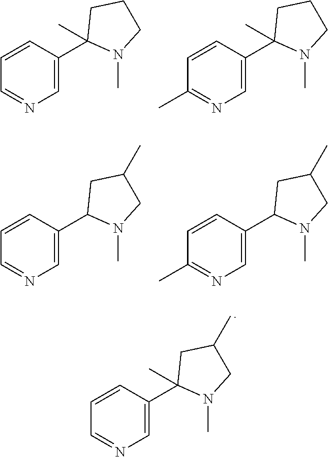

In some embodiments, R⁵ is H, R⁶ is F, CH₃, or OCH₃, R⁷ is H or CH₃, and R⁸ is H or CH₃, provided that at least one of R⁷ and R⁷⁸ is CH₃. Accordingly, in some embodiments, the substituted 3-(1-methylpyrrolidin-2-yl)pyridine has a structure selected from

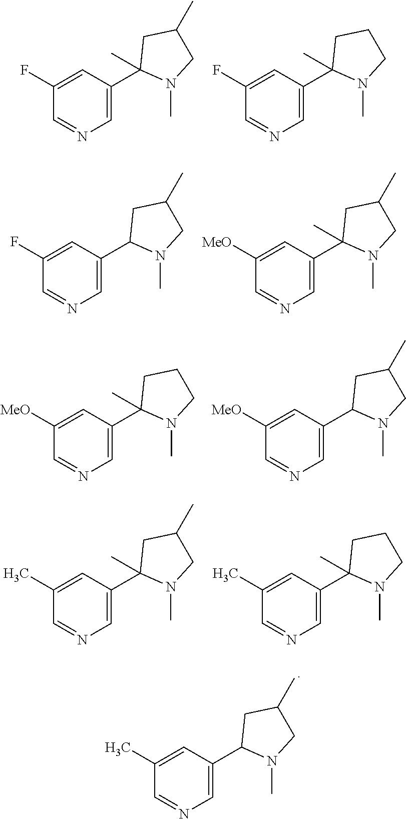

A substituted 3-(1-methylpyrrolidin-2-yl)pyridine and bearing one or more substituents on the pyrrolidine ring as described herein may be present as a single enantiomer or as a mixture of enantiomers. In some embodiments, the substituted 3-(1-methylpyrrolidin-2-yl)pyridine bearing one or more substituents on the pyrrolidine ring is present in racemic form, meaning there are equal amounts of (R)- and (S)-enantiomers present. In some embodiments, the composition comprises unequal amounts of (R)- and (S)-enantiomer (i.e., is enriched in either the (R)- or (S)-enantiomer. In some embodiments, the composition predominantly comprises the substituted 3-(1-methylpyrrolidin-2-yl)pyridine bearing one or more substituents on the pyrrolidine ring in the (R)-configuration, for example, about 90% or more of the total quantity of substituted 3-(1-methylpyrrolidin-2-yl)pyridine bearing one or more substituents on the pyrrolidine ring present is in the (R)-configuration. In some embodiments, the composition predominantly comprises the substituted 3-(1-methylpyrrolidin-2-yl)pyridine bearing one or more substituents on the pyrrolidine ring in the (S)-configuration, for example, about 90% or more of the total quantity of substituted 3-(1-methylpyrrolidin-2-yl)pyridine present is in the (S)-configuration. In some embodiments, the composition comprises 95% or more of the (S)-configuration of the substituted 3-(1-methylpyrrolidin-2-yl)pyridine, based on the total amount of substituted 3-(1-methylpyrrolidin-2-yl)pyridine present.

In some embodiments, the substituted 3-(1-methylpyrrolidin-2-yl)pyridine bearing one or more substituents on the pyrrolidine ring is non-racemic, and has one of the following structures:

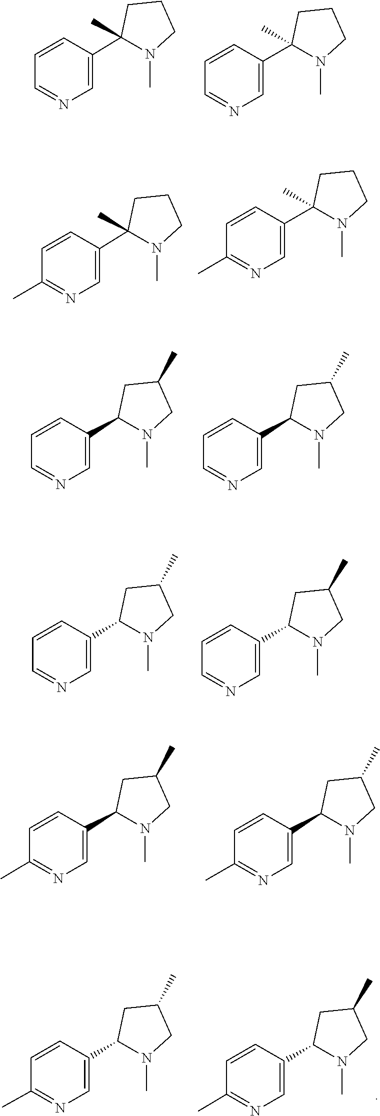

In some embodiments, the substituted 3-(1-methylpyrrolidin-2-yl)pyridine bearing one or more substituents on the pyrrolidine ring is non-racemic, and has a structure selected from:

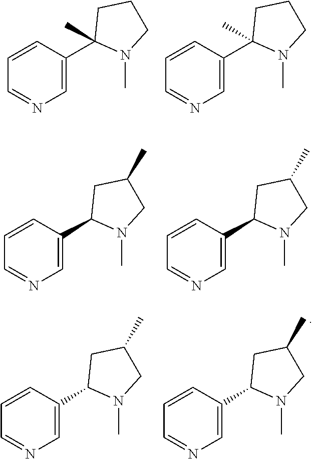

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of various substituted 3-(1-methylpyrrolidin-2-yl)pyridines such as those described herein has been reported in, for example, Lin et al., J. Med. Chem., 1994, 37, 3542-3553; Dukat et al. European Journal of Medicinal Chemistry, 31(11), 1996, 875-888; U.S. Pat. No. 9,440,948; Wang et al., Drug Dev Res 1998, 45(1), 10-16 (each of which is incorporated herein by reference), among many others. Generally, small substituents such as methyl groups are well tolerated at the 2′ or 4′ positions of the nicotine pyrrolidine ring, and small substituents such as alkyl, halogen, alkoxy, and the like are well tolerated at the 5 or 6 position of the nicotine pyridine ring. For example, 3-(1-methylpyrrolidin-2-yl)pyridines bearing a methyl substituent at the 2′ or 4′ position are equipotent or even more potent than nicotine with respect to binding affinity to the nicotinic acetylcholine receptor, and are expected to preserve the pharmacological effects of nicotine in vivo. See, for example U.S. Pat. No. 5,278,176, Lin et al., J. Med. Chem., 1994, 37, 3542-3553, and Wang et al., Drug Dev Res 1998, 45(1), 10-16.

Without wishing to be bound by any particular theory, it is believed that certain substitutions for hydrogen on the pyridine ring and/or pyrrolidine rings of the substituted 3-(1-methylpyrrolidin-2-yl)pyridine compound preserve the general pharmacological profile and physiological effects of nicotine while offering the potential for one or more of greater potency, longer half-life, reduced product consumption, more rapid and/or complete absorption, greater bioavailability, and the like. Particularly, it is believed that in some embodiments, substituted 3-(1-methylpyrrolidin-2-yl)pyridines of the disclosure are readily absorbed through membranes such as pulmonary alveoli, oral mucosa, and the like by virtue of their lipophilicity. Lipophilicity is conveniently measured in terms of log P, the partition coefficient of a molecule between a lipophilic phase and an aqueous phase, usually octanol and water, respectively. Accordingly, in some embodiments, the substituted 3-(1-methylpyrrolidin-2-yl)pyridine of Formula I or II has a calculated or experimental log P of about 1 or greater, where log P is the log₁₀ of the partitioning coefficient of the substituted 3-(1-methylpyrrolidin-2-yl)pyridine between octanol and water. Log P values may be measured experimentally according to protocols well known to one of skill in the art. Alternatively, log P values may be calculated using commercially available software. In some embodiments, the substituted 3-(1-methylpyrrolidin-2-yl)pyridine of Formula I or II has a calculated log P from 1 to about 2, such as 1.1, 1.2, 1.3, 1.4, 1.5, 1.6, 1.7, 1.8, or 1.9. In some embodiments, the substituted 3-(1-methylpyrrolidin-2-yl)pyridine of Formula I or II has a calculated log P from about 1.2 to about 1.7. In some embodiments, the substituted 3-(1-methylpyrrolidin-2-yl)pyridine having a calculated log P from about 1.2 to about 1.7 bears one or more lipophilic substituents on the pyridine ring, such as C₁-C₆ alkyl, or C₁-C₃ alkyl. In some embodiments, the lipophilic substituent is methyl, and is present at the 2, 4, 5, or 6 position of the pyridine ring. In some embodiments, the lipophilic substituted 3-(1-methylpyrrolidin-2-yl)pyridine is 2-methyl-5-(1-methylpyrrolidin-2-yl)pyridine.

The quantity of substituted 3-(1-methylpyrrolidin-2-yl)pyridine (e.g., 2-methyl-5-(1-methylpyrrolidin-2-yl)pyridine) present in the aerosol generating composition may vary. Typically, the substituted 3-(1-methylpyrrolidin-2-yl)pyridine (e.g., 2-methyl-5-(1-methylpyrrolidin-2-yl)pyridine, calculated as the free base) is present in a concentration of at least about 0.001% by weight of the composition, such as in a range from about 0.01% to about 10%. In some embodiments, the substituted 3-(1-methylpyrrolidin-2-yl)pyridine (e.g., 2-methyl-5-(1-methylpyrrolidin-2-yl)pyridine) is present in a concentration from about 0.1% w/w to about 10% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the composition. In some embodiments, the substituted 3-(1-methylpyrrolidin-2-yl)pyridine (e.g., 2-methyl-5-(1-methylpyrrolidin-2-yl)pyridine) is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the composition. In some embodiments, the aerosol generating composition comprises 2-methyl-5-(1-methylpyrrolidin-2-yl)pyridine in an amount from about 0.01 to about 1% by weight, such as from about 0.1 to about 0.75% by weight, based on the total weight of the composition.

The substituted 3-(1-methylpyrrolidin-2-yl)pyridine (e.g., 2-methyl-5-(1-methylpyrrolidin-2-yl)pyridine) may be present in the aerosol generating composition as the free base, as a salt with a suitable acid, in the form of an ion pair with an organic acid, or a combination thereof. Each of these forms is described further herein below.

### 3-(azetidin-2-yl)pyridines and 3-(azetidin-2-ylmethoxy)pyridines

In some embodiments, the aerosol generating material of the disclosure comprises an optionally substituted 3-(azetidin-2-yl)pyridine or an optionally substituted 3-(azetidin-2-ylmethoxy)pyridine. As used herein, the term “substituted 3-(azetidin-2-yl)pyridine” refers to a compound having a 3-(azetidin-2-yl)pyridine scaffold and bearing one or more non-hydrogen substituents on the azetidine ring, and optionally on the pyridine ring. As used herein, the term “substituted 3-(azetidin-2-ylmethoxy)pyridine” refers to a compound having a 3-(azetidin-2-ylmethoxy)pyridine scaffold and bearing one or more non-hydrogen substituents on the azetidine ring, and optionally on the pyridine ring.

In some embodiments, the 3-(azetidin-2-yl)pyridine or 3-(azetidin-2-ylmethoxy)pyridine has a structure according to Formula III:

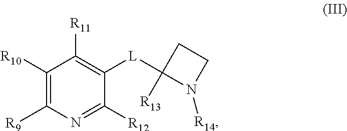

wherein:
L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

In some embodiments, Lis a bond.

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
R¹³ and R¹⁴ are both CH₃;
R¹³ is H and R¹⁴ is CH₃; or
R¹³ is CH₃ and R¹⁴ is H.

In some embodiments, the 3-(azetidin-2-yl) pyridine is 3-(azetidin-2-yl)pyridine, and has a structure:

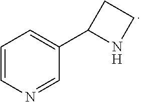

The compound 3-(azetidin-2-yl)pyridine is known in the literature. The synthesis of this compound has been reported in JOC 1979, 44(18), 3136; Med Chem Res (1993) 2:552-5633; in International Patent Application Publication No. WO2012/031220, and in U.S. Pat. Nos. 4,163,855 and 4,163,856, all of which are incorporated herein in their entireties.

In some embodiments, the 3-(azetidin-2-yl) pyridine is 3-(1-methylazetidin-2-yl)pyridine, having the structure:

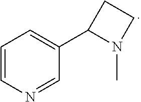

The compound 3-(1-methylazetidin-2-yl)pyridine is known in the literature. The synthesis of this compound has been reported in International Patent Application Publication No. WO2012/031220, previously incorporated by reference herein.

In some embodiments, the 3-(azetidin-2-yl) pyridine has a structure selected from the group consisting of:

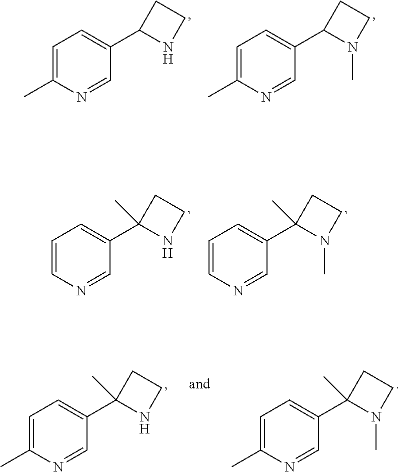

Such compounds are either known, or may be readily prepared according to adaptations of methods utilized for preparation of related 3-(azetidin-2-yl) pyridines and 3-(1-methylpyrrolidin-2-yl)pyridines described herein above. See, e.g., U.S. Pat. No. 4,163,855, previously incorporated by reference herein. The compound 5-(2-azetidinyl)-2-methylpyridine is known in the literature and has a Chemical Abstracts Registry (CAS) Number of 1270467-65-6, and the R- and S-enantiomers have CAS numbers 1213081-15-2 and 1212969-96-4, respectively.

In some embodiments, the composition comprises a 3-(azetidin-2-ylmethoxy)pyridine (i.e., L is —OCH₂—*).

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
R¹³ and R¹⁴ are both CH₃;
R¹³ is H and R¹⁴ is CH₃; or
R¹³ is CH₃ and R¹⁴ is H.

In some embodiments, the 3-(azetidin-2-ylmethoxy)pyridine has a structure selected from the group consisting of:

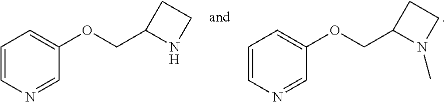

These compounds are known in the literature. The synthesis of these compounds has been reported in International Patent Application Publication No. WO2012/031220, previously incorporated by reference herein.

In some embodiments, the 3-(azetidin-2-ylmethoxy)pyridine has a structure selected from the group consisting of:

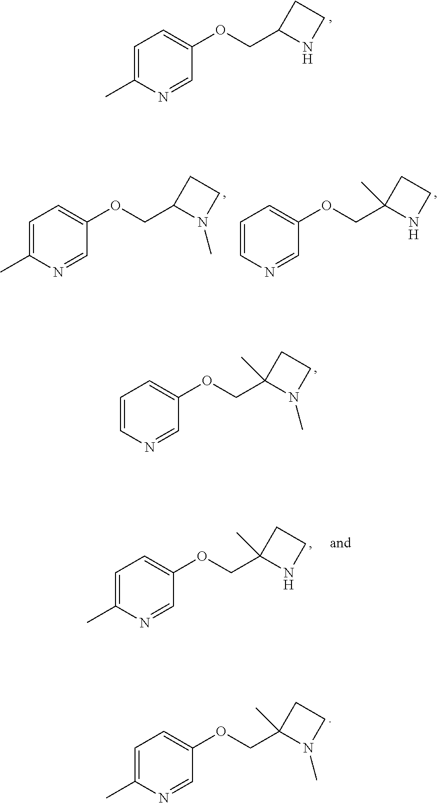

Such compounds are either known, or may be readily prepared according to adaptations of methods utilized for preparation of related the 3-(azetidin-2-ylmethoxy)pyridine, and/or the 3-(azetidin-2-yl)pyridines and substituted 3-(1-methylpyrrolidin-2-yl)pyridines described herein above.

An optionally substituted 3-(azetidin-2-yl)pyridine or optionally substituted 3-(azetidin-2-ylmethoxy)pyridine as described herein may be present as a single enantiomer or as a mixture of enantiomers. In some embodiments, the optionally substituted 3-(azetidin-2-yl)pyridine or optionally substituted 3-(azetidin-2-ylmethoxy)pyridine is present in racemic form, meaning there are equal amounts of (R)- and (S)-enantiomers present. In some embodiments, the composition comprises unequal amounts of (R)- and (S)-enantiomer (i.e., is enriched in either the (R)- or (S)-enantiomer). In some embodiments, the composition predominantly comprises the optionally substituted 3-(azetidin-2-yl)pyridine or optionally substituted 3-(azetidin-2-ylmethoxy)pyridine in the (R)-configuration, for example, about 90% or more of the total quantity of optionally substituted 3-(azetidin-2-yl)pyridine or optionally substituted 3-(azetidin-2-ylmethoxy)pyridine present is in the (R)-configuration. In some embodiments, the composition predominantly comprises the optionally substituted 3-(azetidin-2-yl)pyridine or optionally substituted 3-(azetidin-2-ylmethoxy)pyridine in the (S)-configuration, for example, about 90% or more of the total quantity of optionally substituted 3-(azetidin-2-yl)pyridine or optionally substituted 3-(azetidin-2-ylmethoxy)pyridine present is in the (S)-configuration. In some embodiments, the composition comprises 95% or more of the (S)-configuration of the optionally substituted 3-(azetidin-2-yl)pyridine or optionally substituted 3-(azetidin-2-ylmethoxy)pyridine, based on the total amount of optionally substituted 3-(azetidin-2-yl)pyridine or optionally substituted 3-(azetidin-2-ylmethoxy)pyridine present.

In some embodiments, the optionally substituted 3-(azetidin-2-yl)pyridine is non-racemic, and has one of the following structures:

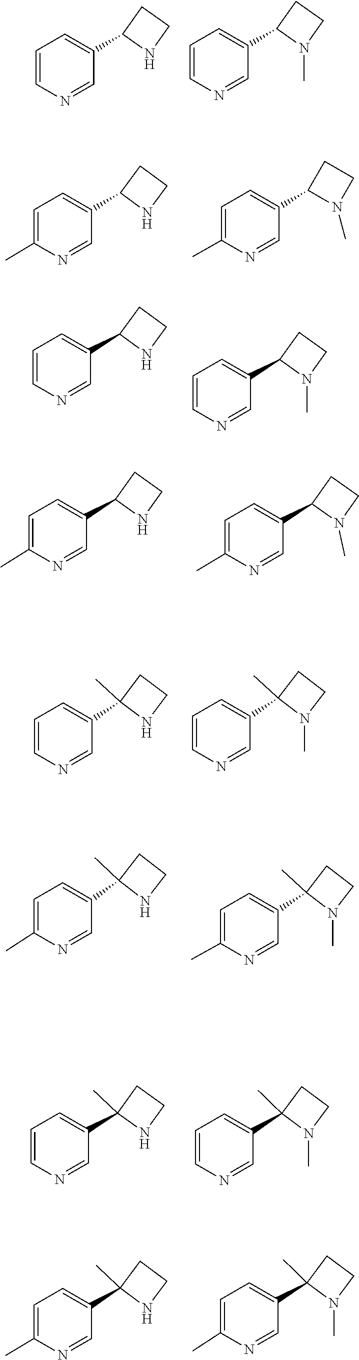

In some embodiments, the optionally substituted 3-(azetidin-2-ylmethoxy)pyridine is non-racemic, and has one of the following structures:

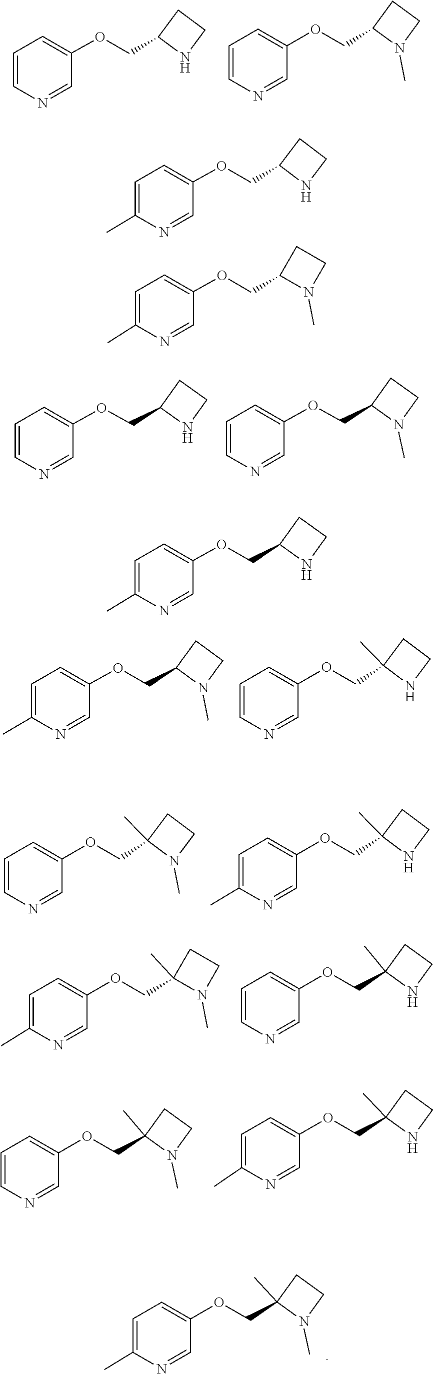

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of certain 3-(azetidin-2-yl)pyridines and 3-(azetidin-2-ylmethoxy)pyridines has been previously disclosed, for example, in the references cited herein with respect to synthesis of such compounds. Generally, these compounds exhibit high affinity for one or more subtypes of nicotinic acetylcholine receptors, particularly the α4β2subtype. The overall pharmacological profiles have been shown to be or are expected to be comparable to that of nicotine.

The quantity of optionally substituted 3-(azetidin-2-yl)pyridine or 3-(azetidin-2-ylmethoxy)pyridine present in the aerosol generating material may vary. Typically, the optionally substituted 3-(azetidin-2-yl)pyridine or 3-(azetidin-2-ylmethoxy)pyridine, calculated as the free base, is present in a concentration of at least about 0.001% by weight of the aerosol generating material, such as in a range from about 0.01% to about 10%. In some embodiments, the optionally substituted 3-(azetidin-2-yl)pyridine or 3-(azetidin-2-ylmethoxy)pyridine is present in a concentration from about 0.05% w/w to about 5% by weight, such as, e.g., from about from about 0.05% w/w. about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the aerosol generating material. In some embodiments, the optionally substituted 3-(azetidin-2-yl)pyridine or 3-(azetidin-2-ylmethoxy)pyridine is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the aerosol generating material. One of skill in the art will recognize that the amount of any particular optionally substituted 3-(azetidin-2-yl)pyridine or 3-(azetidin-2-ylmethoxy)pyridine present in the aerosol generating material may vary based on the potency of the compound, the aerosol generating material matrix, and the desired physiological effect for the aerosol generating material.

In some embodiments, the amount of optionally substituted 3-(azetidin-2-yl)pyridine or 3-(azetidin-2-ylmethoxy)pyridine in the aerosol generating material is determined by potency relative to nicotine. For example, in some embodiments, the amount of optionally substituted 3-(azetidin-2-yl)pyridine or 3-(azetidin-2-ylmethoxy)pyridine is based on the potency factor as described above for substituted 3-(1-methylpyrrolidin-2-yl)pyridines. In some embodiments, an optionally substituted 3-(azetidin-2-yl)pyridine or 3-(azetidin-2-ylmethoxy)pyridine of the disclosure has a potency factor from about 0.1 to about 30, such as from about 2 to about 30.

In some embodiments, the amount of optionally substituted 3-(azetidin-2-yl)pyridine or 3-(azetidin-2-ylmethoxy)pyridine in the aerosol generating material is 1 nicotine equivalent. Accordingly, in some embodiments, 1 nicotine equivalent of optionally substituted 3-(azetidin-2-yl)pyridine or 3-(azetidin-2-ylmethoxy)pyridine is an amount by weight from about 10 to about 0.03 times that of nicotine. In some embodiments, 1 nicotine equivalent of optionally substituted 3-(azetidin-2-yl)pyridine or 3-(azetidin-2-ylmethoxy)pyridine is an amount by weight from about 0.5 to about 0.03 times that of nicotine.

For example, a product comprising 2 mg of nicotine, when the nicotine is replaced by an optionally substituted 3-(azetidin-2-yl)pyridine or 3-(azetidin-2-ylmethoxy)pyridine of the disclosure, may include from about 0.06 mg to about 1 mg of the optionally substituted 3-(azetidin-2-yl)pyridine or 3-(azetidin-2-ylmethoxy)pyridine. Similarly, a product comprising 20 mg of nicotine, when the nicotine is replaced by an optionally substituted 3-(azetidin-2-yl)pyridine or 3-(azetidin-2-ylmethoxy)pyridine, may contain from about 0.6 mg to about 100 mg of the optionally substituted 3-(azetidin-2-yl)pyridine or 3-(azetidin-2-ylmethoxy)pyridine.

In some embodiments, the optionally substituted 3-(azetidin-2-yl)pyridine has the structure:

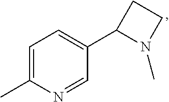

having a potency factor of about 30, meaning that in some embodiments, the amount of optionally substituted 3-(azetidin-2-yl)pyridine present may be about 3% of the amount of nicotine required to achieve the same effect.

In some embodiments, the optionally substituted 3-(azetidin-2-ylmethoxy)pyridine has the structure:

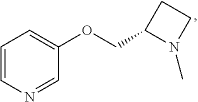

having a potency factor of about 3, meaning that in some embodiments, the amount of 3-(azetidin-2-ylmethoxy)pyridine present may be about 33% of the amount of nicotine required to achieve the same effect.

The optionally substituted 3-(azetidin-2-yl)pyridine or 3-(azetidin-2-ylmethoxy)pyridine may be present in the composition as the free base, as a salt with a suitable acid, or in the form of an ion pair with an organic acid. Each of these forms is described further herein below.

### Other Active Agents

In some embodiments, the substituted 3-(1-methylpyrrolidin-2-yl)pyridine, optionally substituted 3-(azetidin-2-yl)pyridine, or optionally 3-(azetidin-2-ylmethoxy)pyridine of the present disclosure is replaced with, or combined with, other active agents that provide the same general pharmacological profile and/or physiological effects of nicotine. Certain of these active agents may be equipotent or even more potent than nicotine with respect to binding affinity to the nicotinic acetylcholine receptor and are expected to preserve the pharmacological effects of nicotine in vivo. Without wishing to be bound by any particular theory, in some embodiments, these compounds are believed to provide the general pharmacological profile and physiological effects of nicotine while offering the potential for one or more of greater potency, reduced product consumption, more rapid and/or complete absorption, and the like.

Example active agents of this type include, without limitation, cytisine, varenicline, acetylcholine, choline, epibatidine, lobeline, analogs thereof, or combinations thereof. Suitable analogs include any of the above-noted compounds having one or more substituents on any of the carbon atoms thereof, with example substituents including alkyl (e.g., C₁-C₃ alkyl), alkoxy, cycloalkyl, alkenyl, alkynyl, aryl, alkylaryl, amino, halogen, and cyano.

In some embodiments, the other active agent is cytisine or an analog thereof. Cytisine is a naturally occurring alkaloid present in certain plant genera, such as Laburnum and Cytisus of the family Fabaceae. Cytisine (CAS Registry No. 485-35-8) has the structure:

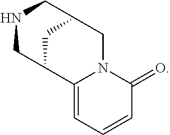

Cytisine is commercially available and has been utilized in post-Soviet states for more than 40 years as an aid to smoking cessation under the brand name Tabex (Sopharma AD). Cytisine is a partial agonist of the α₄β₂ nicotinic acetylcholine receptor.

In some embodiments, the other active agent is varenicline or an analog thereof. Varenicline is commercially available as Chantix® (Pfizer) and is a medication used as an aid for smoking cessation. Varenicline (CAS Registry No. 249296-44-4) has the structure:

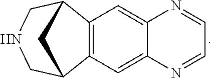 [SMILES: c1cnc2cc3c(cc2n1)[C@@H]1CNC[C@H]3C1]

Like cytisine, varenicline is a partial agonist of the α₄β₂ nicotinic acetylcholine receptor.

The quantity of the other active agent present in the composition may vary. Typically, the other active agent, calculated as the free base, is present in a concentration of at least about 0.001% by weight of the composition, such as in a range from about 0.01% to about 10%. In some embodiments, the other active agent is present in a concentration from about 0.05% w/w to about 5% by weight, such as, e.g., from about from about 0.05% w/w. about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the composition. In some embodiments, the other active agent is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the composition. One of skill in the art will recognize that the amount of any particular other active agent present in the composition may vary based on the potency of the compound, the composition matrix, and the desired physiological effect for the composition.

The other active agent may be present in the aerosol generating material as the free base, as a salt with a suitable acid, or in the form of an ion pair with an organic acid. Each of these forms is described further herein below.

### Free Base

In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active agent exhibits sufficient stability, aqueous solubility, and oral bioavailability such that the free base is suitable for inclusion in the aerosol generating material. Accordingly, in some embodiments, the substituted 3-(1-methylpyrrolidin-2-yl)pyridine, optionally substituted 3-(azetidin-2-yl)pyridine, optionally 3-(azetidin-2-ylmethoxy)pyridine or other active agent is present substantially or completely as the free base. In such embodiments, one of skill in the art will recognize that the aerosol generating material is substantially free of acidic components. By “substantially free” it is meant that no acidic component (e.g., inorganic acid, organic acid, or acids capable of salt has been intentionally added, beyond trace amounts that may be present e.g., as an impurity in another component, or small amounts which may be present in certain flavor packages. For example, some embodiments can have less than 0.001% by weight of any acid component, or less than 0.0001%, or even 0% by weight of any acid component, based on the total weight of the aerosol generating material. In some embodiments, the aerosol generating material is completely free of any acid component (i.e., having 0% or having an amount below the limit of detection). In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active agent is present in the free base form and is adsorbed in a carrier such as a microcrystalline cellulose material to form an adsorption complex.

### Salt

In some embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active agent can be employed in the form of a salt. A “salt” of such compounds is a form characterized by interaction between the said compound in ionic form and a coformer in ionic form (e.g., an acid) via the transfer of one or more protons from the coformer donor to the compound acceptor. The structure of substituted 3-(1-methylpyrrolidin-2-yl)pyridines, optionally substituted 3-(azetidin-2-yl)pyridines, and optionally 3-(azetidin-2-ylmethoxy)pyridines as disclosed herein are such that they comprise two nitrogen atoms that are capable of accepting protons from a coformer and, accordingly, can be present in non-protonated, mono-protonated, and/or di-protonated form in a given sample. Salts of substituted 3-(1-methylpyrrolidin-2-yl)pyridine, optionally substituted 3-(azetidin-2-yl)pyridine, optionally 3-(azetidin-2-ylmethoxy)pyridine, or other active agent can be provided using the types of ingredients and techniques set forth for nicotine in U.S. Pat. No. 2,033,909 to Cox et al. and Perfetti, Beitrage Tabakforschung Int., 12: 43-54 (1983), which are incorporated herein by reference Suitable salts are generally water soluble. Suitable acids for formation of salts (mono- and di-) include, but are not limited to, acetic acid, adipic acid, ascorbic acid, capric acid, citric acid, D-glucuronic acid, D-gluconic acid, lactic acid, galactaric acid, hippuric acid, hydrochloric acid, L-aspartic acid, L-glutamic acid, L-glutaric acid, glycerophosphoric acid, glycolic acid, lauric acid, DL-malic acid, L-malic acid; tartaric acid, palmitic acid, phosphoric acid, sebacic acid, stearic acid, succinic acid, sulfuric acid, thiocyanic acid, (+)-camphoric acid, 1,5-naphthalenedisulfonic acid, 1-hydroxy-2-naphthoic, 2,5-dihydroxybenzoic acid, benzenesulfonic acid, benzoic acid, caprylic acid, cyclamic acid, ethanesulfonic acid, fumaric acid, D-glucoheptonic acid, 4-hydroxybenzoic acid, isobutyric acid, ketoglutaric acid, 2-ketobutyric acid, lactobionic acid, maleic acid, malonic acid, methanesulfonic acid, naphthalene-2-sulfonic acid, nicotinic acid, oleic acid, orotic acid, oxalic acid, pamoic acid, pivalic acid, propionic acid, L-pyroglutamic acid, p-toluenesulfonic acid, (1S)-camphor-10-sulfonic acid, 4-acetamidobenzoic acid, 4-aminosalicylic acid, N-acetyl-4-aminosalicylic acid, caproic acid, dichloroacetic acid, hydrobromic acid, DL-mandelic acid, L-mandelic acid, nitric acid, formic acid, salicylic acid, cinnamic acid, undecylenic acid, isothionic acid, lauric acid, 2-hydroxybenzoic acid, trans-2-hexanoic acid, trimesic acid, 5-nitroisophthalic acid and zinc chloride monohydrate (forming a hydrated zinc chloride complex salt). In some embodiments, the salt is with an organic acid as described herein below.

In some embodiments, a hydrophilic acid is chosen so as to increase water solubility and/or decrease lipophilicity of the salt. Lipophilicity of a salt of a compound as disclosed herein can also be expressed as log D, which is the logarithm of the distribution coefficient, a measure of the pH-dependent differential solubility between an octanol phase and an aqueous phase of all species (ionized and un-ionized) in an octanol/aqueous system, represented by the formula:

$$log {D}_{oct / wat} = log ( \frac{{[ solute ]}_{octanol}}{{[ solute ]}_{water}^{ionized} + {[ solute ]}_{water}^{neutral}} ) .$$

Log D is a commonly used descriptor for the lipophilicity of ionizable compounds. Log D values can be calculated using commercial software or may be determined experimentally in a similar manner to log P but instead of using water, the aqueous phase is adjusted to a specific pH using a buffer. Log D is pH dependent and therefore requires that the pH at which the log D was measured be specified.

When the substituted 3-(1-methylpyrrolidin-2-yl)pyridine, optionally substituted 3-(azetidin-2-yl)pyridine, optionally 3-(azetidin-2-ylmethoxy)pyridine, or other active agent is present in the form of a salt, it is generally preferred that the salt have a relatively low log D, indicative of good water solubility. Without wishing to be bound by theory, it is believed that highly water-soluble salt forms may exhibit a high rate of dissolution, which may be favorable in certain embodiments. Accordingly, in some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active agent salt has a log D from about −1.0 to about 3 at a pH in a range from about 3 to about 11, such as from about −0.5 to about 2, about −0.3 to about 1, or about −0.1 to about 0.

In some embodiments, the selection of acid used to make a salt is performed on the basis of sensory effects of the salt, such as taste. Surprisingly, according to the present disclosure, it has been found that salts of certain organic acids, such as galactaric acid, offer a better taste sensation relative to salts of acids such as tartaric or phthalic acids.

In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active agent is present in the form of a salt with tartaric acid, succinic acid, orotic acid, fumaric acid, pyroglutamic acid, or galactaric acid. In some embodiments, the substituted 3-(1-methylpyrrolidin-2-yl)pyridine, optionally substituted 3-(azetidin-2-yl)pyridine, or optionally 3-(azetidin-2-ylmethoxy)pyridine is present in the form of a salt with succinic acid or galactaric acid.

The stoichiometry of the salts as described herein can vary. For example, in some embodiments, the stoichiometry can range from about 5:1 to about 1:5 molar equivalents of compound:acid, including any whole or fractional value in between. In some embodiments, the molar ratio of compound to acid is about 2:1, about 1:1, or about 1:2. In some embodiments, the compound is present in the form of a bitartrate salt. Hydrates and other solvates of salts are further contemplated herein.

The salts as described herein can, in some embodiments, exist in various polymorphic and pseudopolymorphic forms. Polymorphism is the ability of a crystalline material to exist in more than one form or crystal structure. Polymorphism can result, e.g., from the existence of different crystal packing structures (packing polymorphism) or from the existence of different conformers of the same molecule (conformational polymorphism). Pseudopolymorphism is the result of hydration or solvation of a material and is also referred to as solvomorphism.

### Organic Acid

In some embodiments, the aerosol generating composition comprises an organic acid. In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Further non-limiting examples of organic acids which may be useful in certain embodiments include 2-(4-isobutylphenyl)propanoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid. Examples of suitable acids include, but are not limited to, the list of organic acids in Table 1.

TABLE 1
Non-limiting examples of suitable organic acids
Acid Name

benzoic acid
phenylacetic
p-toluic acid
ethyl benzoic acid
isopropyl benzoic acid
4-phenylbutyric
2-(4-Isobutylphenyl)propanoic acid
2-napthoxyacetic acid
napthylacetic acid
heptanoic acid
octanoic acid
nonanoic acid
decanoic acid
9-deceneoic acid
2-deceneoic acid
10-undecenoic acid
dodecandioic acid
dodecanoic acid
myristic acid
palmitic acid
stearic acid
cyclohexanebutanoic acid
1-heptanesulfonic acid
1-octanesulfonic acid
1-nonanesulfonic acid
monooctyl succinate
tocopherol succinate
monomenthyl succinate
monomenthyl glutarate
norbixin ((2E,4E,6E,8E,10E,12E,14E,16E,18E)-4,8,13,17-
tetramethylicosa-2,4,6,8,10,12,14,16,18-nonaenedioic acid)
bixin ((2E,4E,6E,8E,10E,12E,14E,16Z,18E)-20-methoxy-
4,8,13,17-tetramethyl-20-oxoicosa-2,4,6,8,10,12,14,16,18-
nonaenoic acid)

The selection of organic acid may depend on certain properties. For example, an organic acid should be one recognized as safe for human consumption, and which has acceptable flavor, odor, volatility, stability, and the like. Determination of appropriate organic acids is within the purview of one of skill in the art. In some embodiments, the acid is benzoic acid. In some embodiments, the acid is levulinic acid. In some embodiments, the acid is lactic acid. In some embodiments, the acid is one or more of benzoic acid, lactic acid, and levulinic acid.

In some embodiments, more than one organic acid may be present. For example, the aerosol modifying agent may comprise two, or three, or four, or more organic acids. Accordingly, reference herein to “an organic acid” contemplates mixtures of two or more organic acids. The relative amounts of the multiple organic acids may vary. For example, an aerosol modifying agent may comprise equal amounts of two, or three, or more organic acids, or may comprise different relative amounts. Without wishing to be bound by theory, it is believed that a combination of different organic acids may provide a concentration of any single organic acid in the composition which remains below the threshold which would be found objectionable from a sensory perspective.

The amount of organic acid present in the article (e.g., in the aerosol generating material and/or the body of material), relative to the substituted 3-(1-methylpyrrolidin-2-yl)pyridine, 3-(azetidin-2-yl)pyridine, or 3-(azetidin-2-ylmethoxy)pyridine (e.g., 2-methyl-5-(1-methylpyrrolidin-2-yl)pyridine), may vary. In some embodiments, the aerosol generating material comprises from about 0.05, about 0.1, about 1, about 1.5, about 2, or about 5, to about 10, about 15, or about 20 molar equivalents of the organic acid relative to the substituted 3-(1-methylpyrrolidin-2-yl)pyridine, 3-(azetidin-2-yl)pyridine, or 3-(azetidin-2-ylmethoxy)pyridine, calculated as the free base of the 3-(1-methylpyrrolidin-2-yl)pyridine, 3-(azetidin-2-yl)pyridine, or 3-(azetidin-2-ylmethoxy)pyridine.

In some embodiments, the aerosol generating material comprises from about 2 to about 10, or from about 2 to about 5 molar equivalents of the organic acid relative to the substituted 3-(1-methylpyrrolidin-2-yl)pyridine, 3-(azetidin-2-yl)pyridine, or 3-(azetidin-2-ylmethoxy)pyridine, on a free-base basis. In some embodiments, the organic acid is present in a molar ratio with the substituted 3-(1-methylpyrrolidin-2-yl)pyridine, 3-(azetidin-2-yl)pyridine, or 3-(azetidin-2-ylmethoxy)pyridine from about 2, about 3, about 4, or about 5, to about 6, about 7, about 8, about 9, or about 10. In embodiments wherein more than one organic acid it is to be understood that such molar ratios reflect the totality of the organic acids present.

In some embodiments, the aerosol generating material comprises one or more of the organic acids described herein above. The substituted 3-(1-methylpyrrolidin-2-yl)pyridine, 3-(azetidin-2-yl)pyridine, or 3-(azetidin-2-ylmethoxy)pyridine and the one or more organic acids may be present independently, in the form of a salt, or as a cocrystal. In some embodiments, the aerosol generating material comprises one or more of lactic acid, levulinic acid, benzoic acid, succinic acid, galactaric acid, orotic acid, fumaric acid, pyroglutamic acid, and tartaric acid, and at least a portion of said acid is present in the form of a salt with the substituted 3-(1-methylpyrrolidin-2-yl)pyridine, 3-(azetidin-2-yl)pyridine, or 3-(azetidin-2-ylmethoxy)pyridine.

In some embodiments, the aerosol generating material comprises a lactic acid salt of the substituted 3-(1-methylpyrrolidin-2-yl)pyridine, 3-(azetidin-2-yl)pyridine, or 3-(azetidin-2-ylmethoxy)pyridine.

In some embodiments, the aerosol generating material comprises a levulinic acid salt of the substituted 3-(1-methylpyrrolidin-2-yl)pyridine, 3-(azetidin-2-yl)pyridine, or 3-(azetidin-2-ylmethoxy)pyridine.

In some embodiments, the aerosol generating material comprises a galactaric acid salt of the substituted 3-(1-methylpyrrolidin-2-yl)pyridine, 3-(azetidin-2-yl)pyridine, or 3-(azetidin-2-ylmethoxy)pyridine.

In some embodiments, the aerosol generating material comprises a tartaric acid salt of the substituted 3-(1-methylpyrrolidin-2-yl)pyridine, 3-(azetidin-2-yl)pyridine, or 3-(azetidin-2-ylmethoxy)pyridine.

The stoichiometry of such lactate, levulinate, tartrate, and galactarate salts may vary. Accordingly, the molar ratio of lactic, levulinic, tartaric, or galactaric acid to substituted 3-(1-methylpyrrolidin-2-yl)pyridine, 3-(azetidin-2-yl)pyridine, or 3-(azetidin-2-ylmethoxy)pyridine may be in a range from about 3:1 to about 1:3, including any whole or fractional value in between, such as for example about 2:1, about 1.5:1, about 1:1, about 1:1.5, or about 1:2.

In some embodiments, the aerosol generating material may be substantially or completely free of organic acids (i.e., having less than 0.001% by weight of organic acid, or less than 0.0001%, or even 0% by weight of organic acid, based on the total weight of the aerosol generating material, or as having an amount of organic acid below the limit of detection).

### Resin Complex

In some embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active agent may be present in the form of a polymer complex, where the compound is bound to an acidic polymer. The polymer of such a complex can be any polymer (including homopolymers or all types of copolymers) with acidic functionalities, e.g., a polymeric cation exchange resin. In some embodiments, the polymer comprises acidic sites that can be classified as strongly acidic, weakly acidic, or of intermediate acidity (depending, e.g., on the strength of the acid from which they are derived). In some embodiments, the polymer comprises weakly acidic sites and can be referred to as a weakly acidic cation exchange resin. Non-limiting examples of acidic sites include, e.g., carboxylic acids, sulfonic acids, phosphonous acids, phosphonic acids, phosphoric acids, iminodiacetic acids, and phenolic groups (e.g., as disclosed in Adams et al., J. Soc. Chem. Ind. 54, IT (1935), which is incorporated herein by reference). Suitable polymers include, but are not limited to, addition polymers of styrene and divinylbenzene, divinylbenzene and methacrylic acid, divinylbenzene and acrylic acid, phenolic resins, or cellulose, dextran or pectin cross-linked with, e.g., epichlorohydrin. In some embodiments, the polymer comprises cross-linked moieties. Various acidic ion-exchange resins which are known in the art and are suitable for formation of complexes, include, but are not limited to, polymethacrylic acid resins such as DuPont™ Amberlite™ IRP64, DuPont™ Amberlite™ IRP69, Purolite™ C115HMR, Doshion™ P551, and polyacrylic carbomers, such as Carbopol 974P. See, for example, U.S. Pat. No. 3,901,248 to Lichtneckert et al., which is incorporated herein by reference. In some embodiments, when the substituted 3-(1-methylpyrrolidin-2-yl)pyridine, optionally substituted 3-(azetidin-2-yl)pyridine, or optionally 3-(azetidin-2-ylmethoxy)pyridine is present in the form of a polymer complex, the composition further comprises a divalent metal buffer, such as a calcium or magnesium salt (e.g., carbonate, bicarbonate, oxide, acetate, or the like).

### Cocrystal

In some embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active agent may be present in the form of a co-crystal with at least one other component (“coformer”), both in neutral form. Specifically, as defined in a US FDA industry guidance document, a co-crystal is a solid that is a crystalline material composed of two or more molecules in the same crystal lattice, where the components are in a neutral state and interact via nonionic interactions. See U.S. Department of Health and Human Services, Food and Drug Administration, Guidance for Industry: Regulatory Classification of Pharmaceutical Co-Crystals (April 2013), which is incorporated herein by reference. This form is different and distinct from both salts and ion pairs, each described herein. Specifically, co-crystals can generally be distinguished from salts (and ion pairs) by the absence of a proton transfer between the components (i.e., a substituted 3-(1-methylpyrrolidin-2-yl)pyridine and the one or more coformers) in a co-crystal. The crystalline structure of the co-crystal is generally held together by freely reversible, non-covalent interactions. Co-crystals typically comprise the substituted 3-(1-methylpyrrolidin-2-yl)pyridine, optionally substituted 3-(azetidin-2-yl)pyridine, or optionally 3-(azetidin-2-ylmethoxy)pyridine and coformer in a defined stoichiometric ratio. In some embodiments, co-crystals can encompass hydrates, solvates, and clathrates. Co-crystals can comprise the substituted 3-(1-methylpyrrolidin-2-yl)pyridine, optionally substituted 3-(azetidin-2-yl)pyridine, optionally 3-(azetidin-2-ylmethoxy)pyridine, or other active agent in combination with an organic and/or an inorganic coformer.

Examples of suitable coformers include, but are not limited to, acetamidobenzoic acid, L-proline, tromethamine, urea, xylitol, caffeine, glycine/glycine anhydride, vanillin, methyl 4-hydroxybenzoate(methylparaben), succinimide, L-alanine, mannitol, L-phenylalanine, saccharin, propylparaben, N-methylglucamine, L-tyrosine, gentisic acid, sorbic acid, benzoic acid, L-methionine, maltol, L-lysine, tromethamine, nicotinamide, isonicotinamide, phenylalanine, benzoquinone, terephthalaldehyde, 4-hydroxybenzoic acid, pyruvic acid, 1-hydroxy-2-naphthoic acid, 4-aminobenzoic acid, vanillic acid, ethyl vanillin, isonicotinic acid, gallic acid, menthol (e.g., racemic menthol or (−)-menthol), paracetamol, aspirin, ibuprofen, naproxen, ketoprofen, flurbiprofen, glucose, serine, malic acid, acetamide, sulfacetamide, benzoic acid, creatine, 2-hydroxyethanesulfonic acid, clofibric acid, taurine (tauric acid), iproniazid, L-histadine, L-arginine, L-asparagine, glutamine, L-cysteine, alanine, valine, isoleucine, leucine, morpholine, theronine, N-methylglucamine, 3-hydroxy-2-oxopropionic acid; 2-oxobutyric acid (2-ketobutyric acid), 3-methyl-2-oxobutanoic acid; 3-methyl-2-oxopentanoic acid; 4-methyl-2-oxopentanoic acid; and 2-oxopentanedioic acid, 2-oxo-3-phenylpropionic acid; 5-oxooctanoic acid; and 5-oxodecanoic acid, aldonic acids (e.g., glyceric acid, xylonic acid, gluconic acid, and ascorbic acid), ulosonic acids (e.g., neuraminic acid and ketodeoxyoctulosonic acid), uronic acids (e.g., glucuronic acid, galacturonic acid, and iduronic acid), aldaric acids (e.g., tartaric acid, meso-galactaric acid/mucic acid, and D-glucaric acid/saccharic acid), galactaric acid), and polyfunctional aromatic acids.

In some embodiments, the conformer is a polyfunctional aromatic acid. Polyfunctional aromatic acids often comprise a substituted or unsubstituted phenyl group as the aromatic component, but can alternatively comprise another aromatic moiety, e.g., pyridine, pyrazine, imidazole, pyrazole, oxazole, thiophene, naphthalene, anthracene, and phenanthrene. Substituents on the optionally substituted aromatic acids may be any type of substituent, including, but not limited to, halo (e.g., Cl, F, Br, and I); alkyl, halogenated alkyl (e.g., CF₃, 2-Br-ethyl, CH₂F, CH₂Cl, CH₂CF₃, or CF₂CF₃); alkenyl, hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate groups. Example polyfunctional aromatic acids can be, for example:
substituted and unsubstituted aromatic dicarboxylic acids (e.g., 1,2-benzenedicarboxylic acid (phthalic acid), 1,3-benzenedicarboxylic acid (isophthalic acid), 1,4-benzenedicarboxylic acid (terephthalic acid), 2-iodo-1,3-benzenedicarboxylic acid, 2-hydroxy-1,4-benzenedicarboxylic acid, 2-nitro-1,4-benzenedicarboxylic acid, 3-fluoro-1,2-benzenedicarboxylic acid, 3-amino-1,2-benzenedicarboxylic acid, 3-nitro-1,2-benzenedicarboxylic acid, 4-bromo-1,3-benzenedicarboxylic acid, 4-hydroxy-1,3-benzenedicarboxylic acid, 4-amino-1,2-benzenedicarboxylic acid, 4-nitro-1,2-benzenedicarboxylic acid, 4-sulfo-1,2-benzenedicarboxylic acid, 4-amino-1,3-benzenedicarboxylic acid, 5-bromo-1,3-benzenedicarboxylic acid, 5-hydroxy-1,3-benzenedicarboxylic acid, 5-amino-1,3-benzenedicarboxylic acid, 5-nitro-1,3-benzenedicarboxylic acid, 5-ethynyl-1,3-benzenedicarboxylic acid, 5-cyano-1,3-benzenedicarboxylic acid, 5-nitro-1,3-benzenedicarboxylic acid, 2,5-hydroxy-1,4-benzenedicarboxylic acid, and 2,3,5,6-tetrafluoro-1,4-benzenedicarboxylic acid;
substituted and unsubstituted hydroxybenzoic acids (e.g., 2-hydroxybenzoic acid (salicylic acid), 3-hydroxybenzoic acid, 4-hydroxybenzoic acid, 2-methyl-4-hydroxybenzoic acid, 3-tert-butyl-4-hydroxybenzoic acid, 4-ethoxy-2-hydroxybenzoic acid, 3-chloro-5-hydroxybenzoic acid, 5-chloro-2-hydroxybenzoic acid, 3-bromo-4-hydroxybenzoic acid, 3-bromo-5-hydroxybenzoic acid, 4-bromo-2-hydroxybenzoic acid, 5-bromo-2-hydroxybenzoic acid, 2-fluoro-5-hydroxybenzoic acid, 3-fluoro-4-hydroxybenzoic acid, 3-fluoro-2-hydroxybenzoic acid, 3-fluoro-5-hydroxybenzoic acid, 2-fluoro-6-hydroxybenzoic acid, 4-fluoro-3-hydroxybenzoic acid, 2-fluoro-4-hydroxybenzoic acid, 5-fluoro-2-hydroxybenzoic acid, 2-amino-3-hydroxybenzoic acid, 2-amino-5-hydroxybenzoic acid, 3-amino-2-hydroxybenzoic acid, 3-amino-4-hydroxybenzoic acid, 3-amino-5-hydroxybenzoic acid, 4-amino-2-hydroxybenzoic acid, 4-amino-3-hydroxybenzoic acid, 5-amino-2-hydroxybenzoic acid (mesalamine), 5-aminomethyl-2-hydroxybenzoic acid, 4-formyl-3-hydroxybenzoic acid, 3-formyl-4-hydroxybenzoic acid, 5-(acetylamino)-2-hydroxybenzoic acid), 4-nitro-2-hydroxybenzoic acid, 3,5-diethyl-4-hydroxybenzoic acid, 3,5-di-tert-butyl-4-hydroxybenzoic acid, 3,5-diisopropyl-2-hydroxybenzoic acid, 3,4-dimethoxy-4-hydroxybenzoic acid (syringic acid), 3,5-dichloro-2-hydroxybenzoic acid, 3,5-dichloro-4-hydroxybenzoic acid, 3,6-dichloro-2-hydroxybenzoic acid, 2,3-difluoro-4-hydroxybenzoic acid, 3,4-difluoro-2-hydroxybenzoic acid, 3,5-dibromo-2-hydroxybenzoic acid, 3,5-diodo-2-hydroxybenzoic acid, 4-amino-5-chloro-2-hydroxybenzoic acid, 3,5-dinitro-2-hydroxybenzoic acid, 2,4,6-tribromo-2-hydroxybenzoic acid, 2,3,5,6-tetrafluoro-4-hydroxybenzoic acid, and 2,3,4,5-tetrafluoro-6-hydroxybenzoic acid);
substituted and unsubstituted dihydroxybenzoic acids (e.g., 2,3-dihydroxybenzoic acid (pyrocatechuic acid/hypogallic acid), 2,4-dihydroxybenzoic acid (p-resorcylic acid), 2,5-dihydroxybenzoic acid (gentisic acid/hydroquinonecarboxylic acid), 2,6-dihydroxybenzoic acid (y-resorcylic acid), 3,4-dihydroxybenzoic acid (protocatechuic acid), 3,5-dihydroxybenzoic acid (a-resorcylic acid), 4-hydroxy-3-methoxybenzoic acid (vanillic acid), 6-methyl-2,4-dihydroxybenzoic acid (orsellenic acid), 4-bromo-3,5-dihydroxybenzoic acid, 5-bromo-2,4-dihydroxybenzoic acid, 5-bromo-3,4-dihydroxybenzoic acid, 6-carboxymethyl-2,3-dihydroxybenzoic acid, 3,5-dibromo-2,4-dihydroxybenzoic acid, 3,5-dichloro-2,6-dihydroxybenzoic acid, and 5-amino-3-chloro-2,4-dihydroxybenzoic acid);
substituted and unsubstituted trihydroxybenzoic acids (e.g., 2,3,4-trihydroxybenzoic acid, 2,4,5-trihydroxybenzoic acid, 2,4,6-trihydroxybenzoic acid (phloroglucinol carboxylic acid), and 3,4,5-trihydroxybenzoic acid (gallic acid));
substituted and unsubstituted aromatic tricarboxylic acids (e.g., 1,2,3-benzenetricarboxylic acid, 1,2,4-benzenetricarboxylic acid (trimellitic acid); and
substituted and unsubstituted aromatic tetracarboxylic acids (e.g., 1,2,3,4-benzenetetracarboxylic acid (mellophanic acid) and 1,2,4,5-benzenetetracarboxylic acid (pyromellitic acid). Further contemplated are various combinations of any of the foregoing acids.

In some embodiments, the coformer is L-malic acid, succinic acid, or a combination thereof. In some embodiments, the coformer is 1,1,6,6-tetraphenyl-2,4-hexidiyne-1,6-diol. In some embodiments, the coformer is di-iodotetrafluoro benzene, 4,4′-diiodooctafluorobiphenyl, or 1,4-bis(diphenylhydroxymethyl)benzene. In some embodiments, the coformer is orotic acid.

In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active agent is present in the form of a salt co-crystal. A “salt co-crystal” is a type of hybrid structure with both salt and co-crystal characteristics. Typically, a substituted 3-(1-methylpyrrolidin-2-yl)pyridine, optionally substituted 3-(azetidin-2-yl)pyridine, optionally 3-(azetidin-2-ylmethoxy)pyridine molecule, or other active agent within a salt co-crystal is associated with at least two coformers (which may be the same or different), wherein one coformer is in ionic form (e.g., an acid) and transfers a proton to the substituted 3-(1-methylpyrrolidin-2-yl)pyridine, optionally substituted 3-(azetidin-2-yl)pyridine, optionally 3-(azetidin-2-ylmethoxy)pyridine molecule, or other active agent, and wherein a second coformer does not transfer a proton to the substituted 3-(1-methylpyrrolidin-2-yl)pyridine, optionally substituted 3-(azetidin-2-yl)pyridine, optionally 3-(azetidin-2-ylmethoxy)pyridine molecule, or other active agent. Suitable acids and coformers are generally those described herein above with respect to salts and co-crystals.

The stoichiometry of the co-crystals and salt co-crystals described herein can vary. For example, in certain embodiments, where two components are present, the stoichiometry can range in certain embodiments from about 5:1 to about 1:5 compound:coformer. Where more than one coformer is used to form a co-crystal or salt co-crystal, the ratios of the coformers with respect to both the substituted 3-(1-methylpyrrolidin-2-yl)pyridine, optionally substituted 3-(azetidin-2-yl)pyridine, optionally 3-(azetidin-2-ylmethoxy)pyridine, or other active agent and to one another can also vary.

The co-crystals and salt co-crystals described herein can, in some embodiments, exist in various polymorphic and pseudopolymorphic forms, as well as solvates and hydrates.

In some embodiments, the substituted 3-(1-methylpyrrolidin-2-yl)pyridine, optionally substituted 3-(azetidin-2-yl)pyridine, optionally 3-(azetidin-2-ylmethoxy)pyridine, or other active agent is present in the form of a salt-co-crystal. In some embodiments, the salt-co-crystal is a bis-orotic acid salt-co-crystal. In some embodiments, the bis-orotic acid salt-co-crystal is a hemi-hydrate.

### Ion Pairing

In some embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active agent is present in the form of an ion pair. Ion pairing describes the partial association of oppositely charged ions in relatively concentrated solutions to form distinct chemical species called ion pairs. The strength of the association (i.e., the ion pairing) depends on the electrostatic force of attraction between the positive and negative ions (e.g., a substituted 3-(1-methylpyrrolidin-2-yl)pyridine and the conjugate base of a suitable acid). By “conjugate base” is meant the base resulting from deprotonation of the corresponding acid (e.g., benzoate is the conjugate base of benzoic acid). In embodiments comprising ion pairing, on average, a certain population of these ion pairs exists at any given time, although the formation and dissociation of ion pairs is continuous. In some embodiments, in the composition as disclosed herein, and/or upon oral use of said composition (e.g., upon contact with saliva), the substituted 3-(1-methylpyrrolidin-2-yl)pyridine, optionally substituted 3-(azetidin-2-yl)pyridine, optionally 3-(azetidin-2-ylmethoxy)pyridine, or other active agent and the conjugate base of an acid exist at least partially in the form of an ion pair. Ion pairing is further described in, for example, International Patent Application Publication No. WO2021/050741 to Poole et al., and US Application Publication Nos. 2021/0068447 to Keller et al., 2023/0138306A1 to Zawadzki et al., and 2022/0346434 to Von Cosmos et al., each of which is incorporated herein by reference.

One of skill in the art will recognize that the extent of ion pairing in the disclosed composition, both before and during use by the consumer, may vary based on, for example, pH, the nature of the acid, the concentration of substituted 3-(1-methylpyrrolidin-2-yl)pyridine, optionally substituted 3-(azetidin-2-yl)pyridine, optionally 3-(azetidin-2-ylmethoxy)pyridine, or other active agent, the concentration of the acid or conjugate base of the acid present in the composition, the moisture content of the composition, the ionic strength of the composition, and the like. One of skill in the art will also recognize that ion pairing is an equilibrium process influenced by the foregoing variables. Accordingly, quantification of the extent of ion pairing is difficult or impossible by calculation or direct observation. However, the presence of ion pairing may be demonstrated through surrogate measures, such as partitioning of the substituted 3-(1-methylpyrrolidin-2-yl)pyridine, optionally substituted 3-(azetidin-2-yl)pyridine, optionally 3-(azetidin-2-ylmethoxy)pyridine, or other active agent between octanol and water, or by performing membrane permeation studies of aqueous solutions of, for example, the substituted 3-(1-methylpyrrolidin-2-yl)pyridine plus acids and/or their conjugate bases. An octanol-water partitioning favoring distribution of an ion pair into octanol is predictive of good absorption of the substituted 3-(1-methylpyrrolidin-2-yl)pyridine, optionally substituted 3-(azetidin-2-yl)pyridine, optionally 3-(azetidin-2-ylmethoxy)pyridine (e.g., 2-methyl-5-(1-methylpyrrolidin-2-yl)pyridine), or other active agent through the oral mucosa. However, as described above, in some embodiments, the properties of the substituted 3-(1-methylpyrrolidin-2-yl)pyridine, optionally substituted 3-(azetidin-2-yl)pyridine, optionally 3-(azetidin-2-ylmethoxy)pyridine (e.g., 2-methyl-5-(1-methylpyrrolidin-2-yl)pyridine), or other active agent are such that no ion pairing is required, and accordingly, the composition is substantially or completely free of any ion pairing. By “substantially free” it is meant that no measurable degree of ion pairing is present.

In embodiments where ion pairing is desired, the composition comprises an organic acid, an alkali metal salt of an organic acid, or both. In such embodiments, at least a portion of the substituted 3-(1-methylpyrrolidin-2-yl)pyridine, optionally substituted 3-(azetidin-2-yl)pyridine, optionally 3-(azetidin-2-ylmethoxy)pyridine, or other active agent is associated with at least a portion of the organic acid, the alkali metal salt thereof, or a combination thereof in the form an ion pair. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific composition ingredient as opposed to merely being inherently present as a component of another composition ingredient (e.g., the small amount of organic acid which may inherently be present in a composition ingredient). For the avoidance of doubt, reference herein to an “organic acid” is intended to distinguish the acid present in ion paired forms over the acid which may be present in salts, co-crystal, and salt co-crystals. While one of skill in the art will recognize that certain organic acids suitable for formation of ion pairs overlap with those identified as suitable for salt or co-crystal formation, it is to be understood that the particular acid used for each of salts, co-crystals, and ion pairs are to be selected specifically for each such embodiment, and reference herein to an organic acid is specific to acids suitable for ion pairing. Accordingly, the presence in the composition of an organic acid as defined below is to be interpreted solely with respect to ion pairing, even if such organic acid is also suitable for salt formation or co-crystal formation, and the presence of such an organic acid does not imply that a salt or co-crystal is present unless explicitly identified. Further, in embodiments where there is no ion pairing intended, the composition may be characterized as substantially or completely free of organic acids (i.e., having less than 0.001% by weight of organic acid, or less than 0.0001%, or even 0% by weight of organic acid, based on the total weight of the composition, or as having an amount of organic acid below the limit of detection). This is not to be interpreted as meaning that the composition is substantially or completely free of substituted 3-(1-methylpyrrolidin-2-yl)pyridine salts or substituted 3-(1-methylpyrrolidin-2-yl)pyridine co-crystals unless explicitly recited.

The amount of organic acid or alkali metal salt thereof present in the aerosol generating material, relative to the substituted 3-(1-methylpyrrolidin-2-yl)pyridine, optionally substituted 3-(azetidin-2-yl)pyridine, optionally 3-(azetidin-2-ylmethoxy)pyridine (e.g., 2-methyl-5-(1-methylpyrrolidin-2-yl)pyridine), or other active agent, may vary. Generally, as the concentration of the organic acid (or the conjugate base thereof) increases, the percent of substituted 3-(1-methylpyrrolidin-2-yl)pyridine, optionally substituted 3-(azetidin-2-yl)pyridine, optionally 3-(azetidin-2-ylmethoxy)pyridine, or other active agent that is ion paired with the organic acid increases. This typically increases the partitioning of the substituted 3-(1-methylpyrrolidin-2-yl)pyridine, optionally substituted 3-(azetidin-2-yl)pyridine, optionally 3-(azetidin-2-ylmethoxy)pyridine, or other active agent in the form of an ion pair, into octanol versus water as measured by the log P (the log₁₀ of the partitioning coefficient). In some embodiments, the composition comprises from about 0.05, about 0.1, about 1, about 1.5, about 2, or about 5, to about 10, about 15, or about 20 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally 3-(azetidin-2-ylmethoxy)pyridine, or other active agent, calculated as the free base of the substituted 3-(1-methylpyrrolidin-2-yl)pyridine, optionally substituted 3-(azetidin-2-yl)pyridine, optionally 3-(azetidin-2-ylmethoxy)pyridine, or other active agent.

In some embodiments, the aerosol generating material comprises from about 2 to about 10, or from about 2 to about 5 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally 3-(azetidin-2-ylmethoxy)pyridine, or other active agent on a free-base basis. In some embodiments, the organic acid, the alkali metal salt thereof, or the combination thereof, is present in a molar ratio with the substituted 3-(1-methylpyrrolidin-2-yl)pyridine, optionally substituted 3-(azetidin-2-yl)pyridine, optionally 3-(azetidin-2-ylmethoxy)pyridine, or other active agent from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present. In some embodiments, the aerosol generating material comprises benzoic acid and sodium benzoate wherein a total amount of benzoate (i.e., benzoic acid and benzoate) is in a molar ratio in a range from about 3 to about 5 relative to the substituted 3-(1-methylpyrrolidin-2-yl)pyridine, optionally substituted 3-(azetidin-2-yl)pyridine, optionally 3-(azetidin-2-ylmethoxy)pyridine, or other active agent. In some embodiments, the molar ratio of the total amount of benzoate to the substituted 3-(1-methylpyrrolidin-2-yl)pyridine, optionally substituted 3-(azetidin-2-yl)pyridine, optionally 3-(azetidin-2-ylmethoxy)pyridine, or other active agent is about 3.2 or about 4.8.

In some embodiments, the organic acid inclusion is sufficient to provide a pH of from about 4.0 to about 9.0, such as from about 4.5 to about 7.0, or from about 5.5 to about 7.0, from about 4.0 to about 5.5, or from about 7.0 to about 9.0. Reference herein to “a pH” means the pH of an aqueous solution of the aerosol generating material prepared by dissolving or suspending 5 grams of aerosol generating material in 95 grams of water and measuring the pH of the resulting solution with a calibrated pH meter.

In some embodiments, the organic acid inclusion is sufficient to provide a pH of from about 4.5 to about 6.5, for example, from about 4.5, about 5.0, or about 5.5, to about 6.0, or about 6.5. In some embodiments, the desired pH is from about 4.5 to about 6.5, and the organic acid is provided in a quantity sufficient to provide such a pH. In some embodiments, the organic acid is provided in a quantity sufficient to provide a pH of from about 5.5 to about 6.5, for example, from about 5.5, about 5.6, about 5.7, about 5.8, about 5.9, or about 6.0, to about 6.1, about 6.2, about 6.3, about 6.4, or about 6.5.

In some embodiments, a mineral acid (e.g., hydrochloric acid, sulfuric acid, phosphoric acid, or the like), alone or in combination with an organic acid, is added to adjust the pH of the aerosol generating material to the desired value. In some embodiments, a buffer (e.g., a buffer as described herein below) is added to the aerosol generating material to the desired value, and/or to maintain the pH of the aerosol generating material at the desired value.

In some embodiments, the aerosol generating material further comprises a solubility enhancer to increase the solubility of one or more of the organic acid or salt thereof. Suitable solubility enhancers include, but are not limited to, humectants as described herein, such as glycerol or propylene glycol.

### Other Active Agents

In some embodiments, the thin film, the aerosol generating composition, or both comprise an active agent in addition to the substituted 3-(1-methylpyrrolidin-2-yl)pyridine. The additional active agent may be a physiologically active material, which is a material intended to achieve or enhance a physiological response. The active agent may for example be selected from nutraceuticals, nootropics, psychoactives. The active agent may be naturally occurring or synthetically obtained. The active agent may comprise for example nicotine, caffeine, taurine, theanine, vitamins such as B6 or B12 or C, melatonin, cannabinoids, or constituents, derivatives, or combinations thereof. The active agent may comprise one or more constituents, derivatives or extracts of cannabis or another botanical (other than tobacco).

### Nicotine

In some embodiments, the thin film and/or the aerosol generating material comprises nicotine as an active agent. In some embodiments, this nicotine may be derived from the botanical extract included in the thin film. In other embodiments, such as those where the botanical extract is not a tobacco extract, the nicotine is an additional active substance. In some embodiments, the aerosol generating material has a nicotine content of from about 1.5 wt % to about 7 wt % of the thin film (DWB).

In some embodiments, the aerosol generating composition (e.g., the thin film) may comprise from at least about 1.5 wt %, about 2 wt %, about 2.5 wt %, about 3 wt %, about 3.5 wt %, about 4 wt %, about 4.5 wt % or about 5 wt % of nicotine (DWB). The thin film may comprise no more than about 7 wt %, about 6.5 wt %, about 6 wt %, about 5.5 wt %, about 5 wt %, about 4.5 wt %, about 4 wt %, about 3.5 wt % or about 3 wt % of nicotine (DWB). For example, the aerosol generating composition (e.g., the thin film) may comprise from about 2 to about 6 wt %, or from about 4 to about 5 wt % nicotine by weight (DWB).

In some embodiments, the aerosol generating composition (e.g., the thin film) comprises from about 1 wt %, about 1.5 wt % or about 2 wt % to about 6 wt %, about 5 wt %, about 4 wt % or about 3 wt % of nicotine (DWB).

In some embodiments, the thin film or the aerosol generating composition of the disclosure as a whole can be completely free or substantially free of nicotine (3-(1-methylpyrrolidin-2-yl)pyridine). By “substantially free” it is meant that no nicotine has been intentionally added, beyond trace amounts that may be present e.g., as an impurity in another component, including as a minor impurity in the substituted 3-(1-methylpyrrolidin-2-yl)pyridine.

### Cannabinoids

In some embodiments, the active agent comprises one or more cannabinoids. As used herein, the term “cannabinoid” refers to a class of diverse natural or synthetic chemical compounds that acts on cannabinoid receptors (e.g., CB1 and CB2) in cells that alter neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids).

Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof.

In certain embodiments, the cannabinoid is selected from tetrahydrocannabinol (THC), the primary psychoactive compound in cannabis, and cannabidiol (CBD), another major constituent of the plant, but which is devoid of psychoactivity. All of the above compounds can be used in the form of an isolate from plant material or synthetically derived. Certain cannabinoids, including but not limited to CBD and THC, may exist in more than one isomeric form, for example Δ8- and Δ9-THC. Such isomeric forms may be naturally occurring or may be synthetic. For avoidance of doubt, reference within the present disclosure to a “cannabinoid” is intended to be inclusive of any and all isomeric forms thereof.

In some embodiments, the cannabinoid comprises at least tetrahydrocannabinol (THC). In some embodiments, the cannabinoid is tetrahydrocannabinol (THC). In some embodiments, the THC is Δ8-THC. In some embodiments, the THC is Δ9-THC.

In some embodiments, the cannabinoid comprises at least cannabidiol (CBD). In some embodiments, the cannabinoid is cannabidiol (CBD). In some embodiments, the CBD is synthetic CBD. In some embodiments, the CBD is Δ8-CBD. In some embodiments, the CBD is Δ9-CBD.

In some embodiments, the cannabinoid (e.g., CBD) is added to the aerosol generating composition in the form of an isolate. An isolate is an extract from a plant, such as cannabis, where the active material of interest (in this case the cannabinoid, such as CBD) is present in a high degree of purity, for example greater than 95%, greater than 96%, greater than 97%, greater than 98%, or around 99% purity.

In some embodiments, the cannabinoid is an isolate of CBD in a high degree of purity, and the amount of any other cannabinoid in the aerosol generating composition is no greater than about 1% by weight of the aerosol generating composition, such as no greater than about 0.5% by weight of the aerosol generating composition such as no greater than about 0.1% by weight of the aerosol generating composition, such as no greater than about 0.01% by weight of the aerosol generating composition.

The choice of cannabinoid and the particular percentages thereof which may be present within the aerosol generating composition will vary depending upon the desired characteristics of the material.

In some embodiments, the cannabinoid (such as CBD) is present in the aerosol generating composition (e.g., the thin film) in a concentration of at least about 0.001% by weight of the aerosol generating composition, such as in a range from about 0.001% to about 2% by weight of the aerosol generating composition. In some embodiments, the cannabinoid (such as CBD) is present in the aerosol generating composition in a concentration of from about 0.1% to about 1.5% by weight, based on the total weight of the aerosol generating composition. In some embodiments, the cannabinoid (such as CBD) is present in a concentration from about 0.4% to about 1.5% by weight, based on the total weight of the aerosol generating composition.

Alternatively, or in addition to a cannabinoid, the active agent may include a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids. Such compounds can be used in the same amounts and ratios noted herein for cannabinoids.

### Terpenes

Active agents suitable for use in the aerosol generating composition can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

In some embodiments, the terpene is a terpene derivable from a phytocannabinoid producing plant, such as a plant from the stain of the Cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the active agent comprises more than one terpene. For example, the active agent may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

Terpenes and/or cannabinoids may be present as an active agent, as an aerosol former, or as a flavorant. The amount of terpene and/or cannabinoid present may vary accordingly based on their intended purpose.

In some embodiments, the active agent comprises caffeine, melatonin, an amino acid, a vitamin, or combinations thereof. In some embodiments, the active agent comprises taurine, theanine, vitamin B6, B12, or C, melatonin, cannabinoids, or constituents, derivatives, or combinations thereof.

### Botanical Extract

In some embodiments, the aerosol generating composition (e.g., the thin film) comprises one or more botanical materials in the form of one or more botanical extracts. In some embodiments, the botanical extract is a liquid or solid that has been isolated from a botanical material. As used herein, the term “botanical material” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, or other treatment processes capable of altering the chemical nature of the material). For the purposes of the present disclosure, a “botanical material” includes but is not limited to “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). The botanical materials used in the present disclosure may comprise, without limitation, any of the compounds and sources set forth herein, including mixtures thereof.

Non-limiting examples of botanical materials include without limitation acai berry, alfalfa, allspice, annatto seed, apricot oil, ashwagandha, bacopa monniera, baobab, basil, bee balm, beet root, wild bergamot, black pepper, blueberries, borage seed oil, bugleweed, cacao, calamus root, catnip, catuaba, cayenne pepper, Centella asiatica, chaga mushroom, Chai-hu, chamomile, cherry blossom, chervil, chlorophyll, cinnamon, dark chocolate, citrus, cocoa, comfrey leaf and root, gingko biloba, ginseng, goji berries, grape seed, green tea, black tea, black cohosh, cayenne, chamomile, cloves, cocoa powder, cordyceps, cranberry, curcumin, damiana, dandelion, Dorstenia arifolia, Dorstenia odorata, echinacea, eucalyptus, fennel, feverfew, Galphimia glauca, garlic, ginger, ginseng (e.g., Panax ginseng), goldenseal, green tea, grapefruit, Griffonia simplicifolia, guarana, gutu kola, hawthorn, hemp, hibiscus flower, honeybush, hops, jasmine, jiaogulan, Kaempferia parviflora (Thai ginseng), kava, lavender, lemon balm, lemongrass, licorice, Lion's mane, lutein, maca, matcha, Nardostachys chinensis, marjoram, milk thistle, mints (menthe), oolong tea, orange, oregano, papaya, pennyroyal, peppermint, potato peel, primrose, quercetin, red clover, resveratrol, Rhizoma gastrodiae, Rhodiola, Aspalathus linearis (rooibos; red or green), rose essential oil, rosehip, rosemary, sage, clary sage, savory, saw palmetto, Sceletium tortuosum, Schisandra, Silybum marianum, Skullcap, spearmint, Spikenard, spirulina, slippery elm bark, sorghum bran hi-tannin, sorghum grain hi-tannin, Saint John's Wort, sumac bran, terpenes, thyme, tisanes, turmeric, Turnera aphrodisiaca, Uva ursi, valerian, Viola odorata, white mulberry, wild yam root, wintergreen, Withania somnifera, yacon root, yellow dock, yerba mate, and yerba santa.

In some embodiments, thin film comprises from about 10 wt %, about 20 wt %, about 30 wt %, about 40 wt % or about 45 wt % to about 60 wt %, about 55 wt % or about 50 wt % of botanical extract (DWB). For example, the thin film may comprise from about 20 to about 60 wt %, from about 40 to about 55 wt % or from about 45 to about 50 wt % of botanical extract (DWB).

In some embodiments, the botanical extract included in the thin film is an extract of one or more of any of the botanical materials discussed herein.

In some embodiments, the botanical extract is selected from eucalyptus, star anise, cocoa and hemp. In some embodiments, the botanical extract is selected from rooibos and fennel.

A botanical extract may be prepared by processing techniques such as expression (such as juicing or pressing) or solvent extraction. Optionally, the extract is concentrated and/or purified, for example by distillation. In some embodiments, the botanical material is macerated, frequently without heating, to soften and degrade the material prior to extraction. In some embodiments, the botanical extract is an aqueous extract, obtained by extraction with water. Additionally or alternatively, other solvents may be used, including supercritical fluids.

In some embodiments, the botanical extract is a tobacco extract. In some embodiments, the tobacco extract may be an aqueous extract, obtained by extraction with water. The tobacco extract may be an extract from any suitable tobacco, such as single grades or blends, cut rag or whole leaf, including Virginia and/or Burley and/or Oriental. It may also be an extract from tobacco particle ‘fines’ or dust, expanded tobacco, stems, expanded stems, and other processed stem materials, such as cut rolled stems. The extract may be obtained from a ground tobacco or a reconstituted tobacco material.

A conventional aqueous botanical extract will have a solids (non-aqueous) content of up to about 50%. In contrast, the botanical extracts, including tobacco extracts, used in some embodiments of the present disclosure to form a thin film may have a solids (non-aqueous) content of at least about 65%, at least about 70%, at least about 75% or at least about 78%. This means that the extract is more concentrated than conventional extracts and may provide a thin film with a strong and impactful flavor and active content when heated to form a vapor or aerosol. The high solids content in the botanical extract may, in some embodiments, be achieved by using an alcohol such as ethanol as a co-solvent with water.

### Flavoring Agent

In some embodiments, the aerosol generating composition (e.g., the thin film) as described herein comprises a flavoring agent. As used herein, a “flavoring agent” or “flavorant” is any flavorful or aromatic substance capable of altering the sensory characteristics associated with the aerosol generating composition or the vapor produced therefrom. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy.

Flavoring agents may be imitation, synthetic or natural ingredients or blends thereof. Flavoring agents may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents.

Flavorants may further include flavor enhancers, sensorial receptor site activators or stimulators, and trigeminal sensates, As used herein, “trigeminal sensate” refers to a flavoring agent which has an effect on the trigeminal nerve, producing sensations including heating, cooling, tingling, and the like. Non-limiting examples of trigeminal sensate flavoring agents include capsaicin, citric acid, menthol, Sichuan buttons, erythritol, and cubebol.

In some embodiments, the aerosol generating composition comprises a sensate which provides to the user of such aerosol generating composition a cooling effect. Suitable cooling agents include, but are not limited to, menthane, menthone, menthone ketals, menthone glycerol ketals, substituted p-menthanes, acyclic carboxamides, monomenthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N-ethyl-p-menthane-3-carboxamide (WS-3), ethyl ester of N-[[5-methyl-2-(1-methylethyl)cyclohexyl]carbonyl]glycine (WS-5), WS-14, N,2,3-trimethyl-2-isopropyl butanamide (WS-23), WS-27, WS-30, (−)-Menthyloxyethanol (Coolact® 5), WS-NA (FEMA 4693), WS-116 (FEMA 4603), N-ethyl-2,2-diisopropylbutanamide, isopulegol, menthyloxy propane diol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, menthyl glutarate, N,2,3-trimethyl-2-(1-methylethyl)-butanamide, N-ethyl-trans-2-cis-6-nonadienamide, N,N-dimethyl menthyl succinamide, N-(2-hydroxyethyl)-2,3-dimethyl-2-isopropylbutanamide, substituted p-menthanes, substituted p-menthane-carboxamides, 2-isopropanyl-5-methylcyclohexanol, menthyl ethylene glycol carbonate, menthone glycerol ketals (e.g., menthone 1,2-glycerol ketal), menthone (S)-lactic acid ketal, menthyl acetoacetate, 3-1-menthoxypropane-1,2-diol, menthyl lactate, eucalyptus extract, menthol propylene glycol carbonate, menthol ethylene glycol carbonate, menthol glyceryl ether, N-tert-butyl-p-menthane-3-carboxamide, p-menthane-3-carboxylic acid glycerol ester, methyl-2-isopropyl-bicyclo[2.2.1]heptane-2-carboxamide, (1R,2S,5R)—N-(4-(carbamoylmethyl)phenyl)-menthylcarboxamide, 2-[2-(p-menthan-3-yloxy)ethoxy]ethanol, (1R,2R,4R)-1-(2-Hydroxy-4-methylcyclohexyl)ethenone, 2-(p-tolyloxy)-N-(1H-pyrazol-5-yl)-N-((thiophen-2-yl)methyl)acetamide, menthol methyl ether, menthyl pyrrolidone carboxylate, 2,5-dimethyl-4-(1-pyrrolidinyl)-3(2H)-furanone, cyclic a-keto enamines, and cyclotene derivatives (e.g., 3-methyl-2-(1-pyrrolidinyl)-2-cyclopenten-1-one and 5-methyl-2-(1-pyrrolidinyl)-2-cyclopenten-1-one). Other compounds include the alpha-keto enamines disclosed in U.S. Pat. No. 6,592,884 to Hofmann et al., which is incorporated in its entirety herein. These and other suitable cooling agents are further described in the following U.S. patents, all of which are incorporated in their entirety by reference hereto: U.S. Pat. Nos. 4,230,688; 4,032,661; 4,459,425; 4,178,459; 4,296,255; 4,136,163; 5,009,893; 5,266,592; 5,698,181; 6,277,385; 6,627,233; 7,030,273. Still other suitable cooling agents are further described in US Patent Application Publications Nos. 2005/0222256 and 2005/0265930, each of which are incorporated in their entirety by reference hereto. In some embodiments, the cooling agent comprises menthol, eucalyptus, mint, menthol, menthyl esters, eucolyptol, WS-3, WS-23, WS-5, (1R,2S,5R)—N-(4-(cyanomethyl)phenyl)menthylcarboxamide (Evercool™ 180), (1R,2S,5R)—N-(2-(pyridin-2-yl)ethyl)menthylcarboxamide (Evercool™ 190), or a combination thereof.

In some embodiments, the aerosol generating composition does not comprise a flavoring agent, and comprises only a cooling agent to provide the desired user experience. In some embodiments, the cooling agent is WS-3.

In some embodiments, the aerosol generating composition comprises a modulator or sensate which provides to the user of such composition a warming effect. Suitable warming agents include, but are not limited to, ethers of vanillyl alcohol (e.g., methyl, ethyl, n-propyl, isopropyl, n-butyl, isobutyl, isoamyl, n-hexyl), gingerol, shogaol, paradol, zingerone, capsaicin, dihydrocapsaicin, nordihydrocapsaicin, homocapsaicin, homodihydrocapsaicin, benzyl alcohol, and combinations thereof. In some embodiments, the warming agent comprises vanillyl butyl ether, vanillyl ethyl ether, capsaicin, or a combination thereof.

Flavoring agents may be in any suitable form, for example, a liquid such as an oil, or a solid such as a powder or wax. In some instances, the flavoring agent may be provided in a spray-dried form or a liquid form.

The amount of flavoring agent utilized in the aerosol generating composition can vary, but is typically up to about 60% by weight. For example, the thin film may comprise up to about 60 wt %, about 50 wt %, about 40 wt %, about 30 wt %, about 20 wt %, about 10 wt % or about 5 wt % of a flavoring agent. In some embodiments, the thin film may comprise at least about 0.5 wt %, about 1 wt %, about 2 wt %, about 5 wt %, about 10 wt %, about 20 wt % or about 30 wt % of flavoring agent (calculated on a dry weight basis). For example, the thin film may comprise from about 10 to about 60 wt %, from about 20 to about 50 wt % or from about 30 to about 40 wt % of flavoring agent.

In some embodiments, the flavor comprises menthol, spearmint and/or peppermint.

In some embodiments, the flavor comprises flavor components of cucumber, blueberry, citrus fruits and/or redberry.

In some embodiments, the flavor comprises eugenol.

In some embodiments, the flavoring agent (if present) comprises, consists essentially of, or consists of, menthol. In some embodiments, the thin film does not comprise an added flavor.

### Overall Thin Film Composition and Format

In some embodiments, the thin film comprises, in addition to an active agent and aerosol former as disclosed herein, guar gum, ground cellulose, and wood pulp as the binder/filler components. In some embodiments, the thin film comprises guar gum, ground cellulose, wood pulp, and a botanical and/or tobacco extract as disclosed herein. In some embodiments, the thin film comprises, in addition to the active agent: 1-10 wt % guar gum; 10-30 wt % glycerol; 10-60 wt % of a botanical extract; 1-15 wt % wood pulp; and 20-40 wt % ground cellulose; wherein these weights are calculated on a dry weight basis. It has been found according to the disclosure that thin films having these compositions can be efficiently heated to generate an inhalable aerosol.

In some embodiments, the thin film is substantially free from (solid) botanical material. In some embodiments, the thin film is substantially free of solid tobacco material. In some embodiments, the thin film is substantially free of tobacco extract.

In some embodiments, the thin film is formed as a sheet, and may be incorporated into an article as described herein below, as for example, a planar sheet, a bunched or gathered sheet, as a crimped sheet, or as a rolled sheet

In some embodiments, the thin film may have a thickness of from about 0.015 mm to about 1.0 mm. Suitably, the thickness may be in the range of from about 0.05 mm, about 0.1 mm or about 0.15 mm to about 0.5 mm or about 0.3 mm. A material having a thickness of about 0.2 mm may be particularly suitable. The thin film may comprise more than one layer, and the thickness described herein refers to the aggregate thickness of those layers.

If the thin film is too thick, then heating efficiency may be compromised. This adversely affects the power consumption in use. Conversely, if the thin film is too thin, it is difficult to manufacture and handle; a very thin material is harder to cast and may be fragile, compromising aerosol formation in use.

The thin film thicknesses stipulated herein optimizes the material properties in view of these competing considerations. The thickness stipulated herein is a mean thickness for the material. In some embodiments, the thin film thickness may vary by no more than about 25%, about 20%, about 15%, about 10%, about 5% or about 1%.

The thin film may have any suitable area density, such as from about 30 g/m² to about 150 g/m². In some embodiments, the thin film may have an area density of from about 30 to about 70 g/m², or from about 40 to about 60 g/m². In some embodiments, the thin film may have an area density of from about 80 to about 150 g/m², or from about 70 to about 120 g/m², or particularly from about 90 to about 110 g/m². Such area densities may be particularly suitable where the thin film is included in an aerosol generating article/assembly in sheet form, or as a shredded sheet (described further hereinbelow).

In some examples, the thin film in sheet form may have a tensile strength of from about 200 N/m to about 900 N/m. In some examples, such as where the thin film does not comprise a filler, the thin film may have a tensile strength of from about 200 N/m to about 400 N/m, or from about 200 N/m to about 300 N/m, or about 250 N/m. Such tensile strengths may be particularly suitable for embodiments wherein the aerosol-generating material is formed as a sheet and then shredded and incorporated into an aerosol generating article. In some examples, such as where the thin film comprises a filler, the thin film may have a tensile strength of from about 600 N/m to about 900 N/m, or from about 700 N/m to about 900 N/m, or about 800 N/m. Such tensile strengths may be particularly suitable for embodiments wherein the aerosol-generating material is included in an aerosol generating article/assembly as a wrapper or as a rolled sheet, suitably in the form of a tube.

In some embodiments, the aerosol generating composition may additionally comprise a carrier on which the thin film is provided. This carrier may ease manufacture and/or handling through, for example, (a) providing a surface onto which a slurry may be cast (and which the slurry does not need to be separated from later), (b) providing a non-tacky surface for the aerosol-generating material, (c) providing some rigidity to the aerosol generating composition.

In some embodiments, the carrier may be formed from materials selected from metal foil, paper, carbon paper, greaseproof paper, ceramic, carbon allotropes such as graphite and graphene, plastic, cardboard, wood or combinations thereof. In some embodiments, the carrier may comprise or consist of a botanical material, such as a sheet of reconstituted tobacco. In some embodiments, the carrier may be formed from materials selected from metal foil, paper, cardboard, wood or combinations thereof. In some embodiments, the carrier itself be a laminate structure comprising layers of materials selected from the preceding lists. In some embodiments, the carrier may also function as a flavor carrier. For example, the carrier may be impregnated with a flavorant or with botanical extract.

In some embodiments, the carrier may be substantially or wholly impermeable to gas and/or aerosol. This prevents aerosol or gas passage through the carrier in use, thereby controlling the flow and ensuring it is delivered to the user. This can also be used to prevent condensation or other deposition of the gas/aerosol in use on, for example, the surface of a heater provided in an aerosol generating assembly. Thus, consumption efficiency and hygiene can be improved in some embodiments.

In some embodiments, the carrier may comprise or consist of a porous layer that abuts the thin film. For example, the porous layer may be a paper layer. In some particular, the thin film is disposed in direct contact with the porous layer; the porous layer abuts the thin film and forms a strong bond. The thin film is formed by drying a slurry or gel and, without being limited by theory, it is thought that the slurry or gel partially impregnates the porous layer (e.g., paper) so that when the slurry or gel sets and forms cross-links, the porous layer is partially bound into the thin film. This provides a strong binding between the thin film and the porous layer.

Additionally, surface roughness may contribute to the strength of bond between the thin film and the carrier. The paper roughness (for the surface abutting the carrier) may suitably be in the range of from about 50 to about 1000 Bekk seconds, suitably from about 50 to about 150 Bekk seconds, suitably about 100 Bekk seconds (measured over an air pressure interval of 50.66-48.00 kPa). (A Bekk smoothness tester is an instrument used to determine the smoothness of a paper surface, in which air at a specified pressure is leaked between a smooth glass surface and a paper sample, and the time (in seconds) for a fixed volume of air to seep between these surfaces is the “Bekk smoothness”.)

Conversely, the surface of the carrier facing away from the thin film may be arranged in contact with the heater, and a smoother surface may provide more efficient heat transfer. Thus, in some embodiments, the carrier is disposed so as to have a rougher side abutting the thin film and a smoother side facing away from the thin film.

In one particular case, the carrier may be a paper-backed foil; the paper layer abuts the thin film layer and the properties discussed in the previous paragraphs are afforded by this abutment. The foil backing is substantially impermeable, providing control of the aerosol flow path. A metal foil backing may also serve to conduct heat to the thin film.

In another case, the foil layer of the paper-backed foil abuts the thin film. The foil is substantially impermeable, thereby preventing water provided in the thin film from being absorbed into the paper which could weaken its structural integrity.

In some embodiments, the carrier is formed from or comprises metal foil, such as aluminum foil. A metallic carrier may allow for better conduction of thermal energy to the thin film. Additionally, or alternatively, a metal foil may function as a susceptor in an induction heating system. In particular embodiments, the carrier comprises a metal foil layer and a support layer, such as cardboard. In these embodiments, the metal foil layer may have a thickness of no more than about 20 μm, such as from about 1 μm to about 10 μm, suitably about 5 μm.

In some embodiments, the carrier may be magnetic. This functionality may be used to fasten the carrier to the assembly in use, or may be used to generate particular thin film shapes. In some embodiments, the aerosol-generating material may comprise one or more magnets which can be used to fasten the material to an induction heater in use.

### Botanical Material

In some embodiments, the aerosol generating composition comprises, in addition to the thin film as disclosed herein, one or more botanical materials in solid form (e.g., as particulates, shredded, and the like). Suitable botanical materials include those described herein above with respect to botanical extracts, as well as tobacco materials.

In some embodiments, the botanical material comprises leaf material. In some embodiments, the botanical material comprises a shredded non-tobacco botanical material. In some embodiments, the shredded non-tobacco botanical material comprises or is rooibos.

In some embodiments, the botanical material comprises a tobacco material. As used herein, the term “tobacco material” refers to any material comprising tobacco or derivatives thereof. The term “tobacco material” may include one or more of tobacco, tobacco derivatives, expanded tobacco, reconstituted tobacco or tobacco substitutes. The tobacco material may comprise one or more of ground tobacco, tobacco fiber, cut tobacco, extruded tobacco, tobacco stem, reconstituted tobacco and/or tobacco extract.

The tobacco used to produce tobacco material may be any suitable tobacco, such as single grades or blends, cut rag or whole leaf, including Virginia and/or Burley and/or Oriental. It may also be tobacco particle ‘fines’ or dust, expanded tobacco, stems, expanded stems, and other processed stem materials, such as cut rolled stems. The tobacco material may be a ground tobacco or a reconstituted tobacco material. The reconstituted tobacco material may comprise tobacco fibers, and may be formed by casting, a Fourdrinier-based paper making-type approach with back addition of tobacco extract, or by extrusion.

The botanical material may, in some embodiments, comprise water. In some instances, the botanical material is a dried botanical material, that is, the plant material has been treated to remove the water naturally present in the plant. In some embodiments, the botanical material comprises no more than about 20 wt %, about 15 wt %, about 12 wt % or about 10 wt % water. In some embodiments, the botanical material may comprise at least about 1 wt %, about 2 wt % or about 5 wt % water. The botanical may comprise from about 10 wt % to about 20 wt % water, or from about 10 wt % to about 15 wt %. Suitably, the water content of the thin film may be about 12 wt %.

In some embodiments, the botanical material comprises one or more aerosol formers as described herein above. The aerosol former may be added to or incorporated into the botanical material. The aerosol former may be the same or different relative to the aerosol former of the thin film.

In some embodiments, the botanical material comprises at least about 10 wt %, about 12 wt %, about 15 wt %, about 18 wt %, about 20 wt % or about 22 wt % of aerosol former. Additionally or alternatively, the botanical material comprises no more than about 25 wt %, about 22 wt %, about 20 wt %, about 18 wt %, about 15 wt % or about 12 wt % of aerosol former. In some embodiments, the amount of aerosol former in the botanical material is from about 15 wt % to about 25 wt %, or from about 20 wt % to about 25 wt % (DWB).

The relative amounts of the thin film and, when present, the botanical material in the aerosol generating composition may vary. In some embodiments, the aerosol generating composition comprises from about 5 wt % to about 95 wt % of botanical material and from about 95 wt % to about 5 wt % of the thin film. For example, in some embodiments, the aerosol generating composition comprises from about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt % or about 95 wt % of the botanical material or of the thin film. In some embodiments, the aerosol generating composition comprises no more than about 95 wt %, about 90 wt %, about 85 wt %, about 80 wt %, about 75 wt %, about 70 wt %, about 65 wt %, about 60 wt %, about 55 wt %, about 50 wt %, about 45 wt %, about 40 wt %, about 35 wt %, about 30 wt %, about 25 wt %, about 20 wt %, about 15 wt %, about 10 wt % or about 5 wt % of the botanical material or of the thin film.

In some embodiments, the aerosol generating composition comprises from about 50 wt % to about 95 wt % of the botanical material and from about 5 wt % to about 50 wt % of the thin film.

In some embodiments, the aerosol generating composition comprises from about 70 to about 90% of the botanical material and from about 10 wt % to about 30 wt % of the thin film.

The thin film and the botanical material may have different thicknesses and/or may be provided in otherwise different sizes, such as cut to different dimensions or average particle sizes. The thin film and the botanical material may be combined in the aerosol generating composition in different configurations. In some embodiments, the thin film and the botanical material may be mixed to form a largely homogenous blend. In other embodiments, the distribution of the thin film and the botanical material is controlled to provide a desired distribution within the composition, optionally with the materials being unmixed or substantially unmixed. For example, the blend may provide a consistent release of aerosol from the two materials throughout use of the composition, or may be configured/formulated to provide either rapid release, a slow release, or separate release of aerosol from the individual components. For example, the materials may be arranged within the composition to allow them to be heated together or separately. Separate heating may mean in terms of timing, for example, sequentially, and/or in terms of heating to different temperatures.

In some embodiments, the thin film is present as a cut or shredded sheet which is blended with the botanical material.

In some embodiments, the aerosol generating composition comprises a plug or section comprising the botanical material, and the thin film is present in the form of a sheet circumscribing at least a portion of the plug or section.

In some embodiments, the aerosol generating composition further comprises a paper wrapper circumscribing the aerosol generating composition, and the thin film is present in the form of a sheet positioned between the botanical material and the wrapper. The outer paper wrapper provides the article with an attractive and clean appearance, and adequate stiffness. The wrapper may also help to retain the glycerol within the aerosol generating composition.

In some embodiments, the aerosol generating composition comprises a first section comprising the botanical material and a second section comprising the thin film in the form of a planar sheet, as a bunched or gathered sheet, as a crimped sheet, or as a rolled sheet (i.e., in the form of a tube or as a rolled plug).

In some embodiments, the aerosol generating composition comprises a first section comprising the botanical material and a second section comprising the thin film in the form of a rolled sheet.

In some embodiments, the aerosol generating composition further comprises a second thin film, the second thin film comprising a binder, an aerosol former, and optionally, an active agent. In some embodiments, one or more of the binder, the aerosol former, and the active agent are the same as in the first thin film. In some embodiments, one or more of the binder, the aerosol former, and the active agent are different from that present in the first thin film. In some embodiments, the second thin film does not comprise any active agent.

In some embodiments, the aerosol generating composition may comprise heating means embedded in the thin film, such as resistive or inductive heating elements, described further herein below.

### Aerosol Generating Article

In another aspect is provided an aerosol generating article comprising an aerosol generating portion comprising the aerosol generating composition as described herein. The aerosol generating article may include additional features including but not limited to, ventilation, a mouthpiece, filter, and a cooling element.

In some embodiments, the article includes ventilation that allows ambient air to be drawn into the article when the article is in use. It is known for aerosol generating articles or assemblies to comprise ventilation in the form of apertures or a porous section provided to allow ambient air to enter the article via the sidewall. In some embodiments, the ventilation may be provided in mouthpiece section of the article, for example, allowing the ambient air to enter into a cooling element, a filter and/or a mouth end section positioned toward the mouth end of the article and downstream of the aerosol-generating section.

The ventilation may allow cool air to be drawn into the article during use, which can mix with the heated volatilized components, thereby cooling the aerosol.

The ventilation may enhance the generation of visible heated volatilized components from the article when it is heated in use. The heated volatilized components are made visible by the process of cooling the heated volatilized components such that supersaturation of the heated volatilized components occurs. The heated volatilized components then undergo droplet formation, otherwise known as nucleation, and eventually the size of the aerosol particles of the heated volatilized components increases by further condensation of the heated volatilized components and by coagulation of newly formed droplets from the heated volatilized components. Ventilation also helps to slow down the flow of aerosol drawn through the mouthpiece and thereby enables the aerosol to cool sufficiently before it reaches the user.

The properties of the aerosol generating compositions disclosed herein mean that aerosol generating articles can be produced having reduced levels of ventilation whilst still providing an inhalable medium with the desired properties and, in particular, avoiding the phenomenon of hot puff. A reduction in ventilation has benefits as ventilation dilutes the aerosol generated by heating the aerosol-generating composition, thereby reducing the flavor and the active delivery and therefore diminishing the user experience.

In conventional tobacco heating (heat-not-burn) aerosol-generating articles, the ventilation ratio may as high as about 75% based on the volume of the aerosol generated by the aerosol-generating composition and drawn through the article. Indeed, it is challenging to prevent hot puff in a conventional tobacco heating aerosol-generating article with a ventilation ratio as low as about 60%.

The ventilation ratio designates the ratio of ambient air drawn in through the ventilation area and combined with the vapor and/or aerosol being drawn through the aerosol-generating article. Thus, a ventilation ratio of 75% means that the aerosol exiting the aerosol-generating article comprises 75% by volume of ambient air and 25% by volume of the vapor and/or aerosol generated by heating the aerosol-generating composition. The ventilation ratio is measured using ISO 9512:2019.

In some embodiments, the article comprises a ventilation area that permits no more than about 50% ventilation based on the volume of aerosol generated by the aerosol-generating composition passing through the article when the article is heated by an aerosol delivery device. In some embodiments, the ventilation is no more than about 49%, no more than about 48%, no more than about 47%, no more than about 46%, no more than about 45%, no more than about 44%, no more than about 43%, no more than about 42%, no more than about 41%, or no more than about 40%.

The amount of ventilation is selected to ensure that there is no hot puff, whilst providing an aerosol of the desired temperature for user comfort and an aerosol with the desired flavor and/or active delivery. According to the present disclosure, the amount of ventilation may be reduced because the aerosol-generating composition has a relatively low moisture content, which reduces the risk of hot puff.

In some embodiments, the ventilation is provided by ventilation holes or apertures, for example, formed as laser perforations. The ventilation holes may pass though the tipping paper, plug wrap and into the material of the article, such as into a plug or section of the aerosol-generating composition or into the mouthpiece or part thereof.

The ventilation holes may be arranged as one or more rows around the circumference of the aerosol-generating article. One or more of the holes may have the form of a slot.

In some embodiments, the aerosol generating article may be circumscribed by a wrapping material such as a porous paper, and the ventilation is provided by an area surrounded by wrapping material, and preferably around the mouthpiece section of the article. The porous wrapper allows air to enter the mouthpiece of the article to cool the mouthpiece.

The porous wrapper may be configured to be partially blocked by a user's lips during use to control the amount of air entering the mouthpiece section through the ventilation area.

Preferably, the ventilation is positioned downstream of the aerosol-generating material and is spaced from the mouth end of the aerosol-generating article sufficiently to ensure that it is not blocked when the user contacts the article with their lips during use. For example, the ventilation may be positioned from about 17 to about 20 mm from the mouth end of the article.

In some embodiments, the article may comprise a mouthpiece. In some embodiments, the ventilation is located to allow the ambient air to enter the mouthpiece section of the article. The ventilation area may be configured such that when the mouthpiece section is placed between a user's lips and an aerosol generated by the aerosol-generating material is drawn into the user's mouth through mouthpiece section, air is drawn into the mouthpiece section through the ventilation area to cool the mouthpiece section.

The mouthpiece may comprise a filter and/or cooling element. In some embodiments, the filter and/or cooling element and the aerosol-generating portion are joined by tipping paper that circumscribes at least a portion of both of these parts of the article.

The cooling element, if present, may act or function to cool gaseous or aerosol components. In some embodiments, it may act to cool gaseous components such that they condense to form an aerosol. It may also act to space the very hot parts of the apparatus from the user. In some embodiments, the cooling segment comprises a longitudinally extending air channel for cooling the flow of air therethrough.

In some embodiments, the ventilation is provided into the cooling element.

The filter, if present, may comprise any suitable filter known in the art such as a cellulose acetate plug or a paper plug, and optionally including capsule.

In some embodiments, the ventilation is provided into the filter. In such embodiments, the perforations may extend into the plug of filter material to ensure that the ambient air enters the plug of filter material and adequately mixes with the aerosol travelling through the filter plug.

In some embodiments, the article comprises a mouth end hollow tubular body. The one or more sections selected from the mouth end tubular body, filter plug and cooling element may be combined by a wrapping material to form a mouthpiece of the article. The mouthpiece may be attached to the aerosol-generating portion, for example by a tipping paper.

In some embodiments, ventilation is provided into the hollow tubular body, which has been found to be particularly beneficial in assisting with the aerosol generation process.

The aerosol generating material and the mouthpiece, made up of one or more of a cooling element, filter plug and hollow tubular mouth end section, may be axially aligned along a longitudinal axis. Optionally, the one or more sections making up the mouthpiece may be joined by a porous wrapper. The porous wrapper may be exposed by the at least one hole or aperture of the ventilation area.

The aerosol generating material and the mouthpiece may be joined by a wrapper, wherein a portion of the porous wrapper joining the section(s) of the mouthpiece may be exposed by a split in the wrapper.

In some embodiments, one or more sections making up the mouthpiece may comprise a ventilation area configured such that the aerosol flowing through the mouthpiece from the aerosol generating material mixes with air drawn into the mouthpiece via the ventilation area.

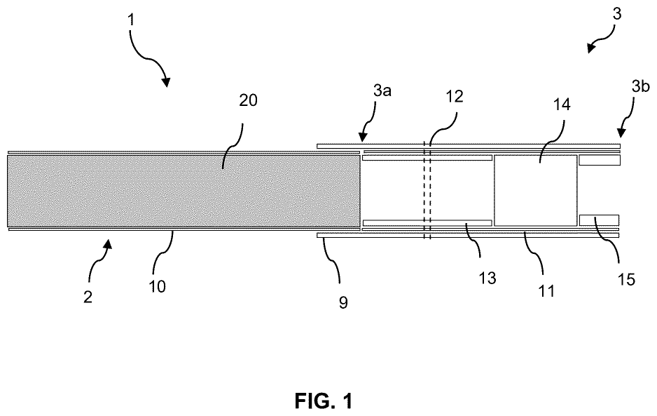

The aerosol generating article according to embodiments of the disclosure may be further illustrated by reference to the non-limiting Figures. FIG. 1 is a side-on cross-sectional view of an article 1 for use in an aerosol provision system. In the present case, the article 1 comprises a consumable for a non-combustible aerosol provision system. With reference to FIG. 1, the article 1 comprises an aerosol generating portion, in the present case a cylindrical aerosol-generating portion 2, and a mouthpiece 3 downstream from and connected to the aerosol-generating portion 2. The aerosol generating portion 2 comprises a rod or segment of aerosol-generating composition 20 wrapped in a rod wrapper 10.

In FIG. 1, the rod of aerosol-generating composition 20 comprises a blend of a first aerosol-generating material comprising botanical material and a second aerosol-generating material comprising a thin film. In this embodiment, the two materials are cut or shredded and the two different shredded materials are blended and formed into a rod segment. In this embodiment, the two aerosol-generating materials are fairly evenly distributed within the rod and along the length of the rod. The article 1 also comprises a mouthpiece 3 which has a mouth end 3b and a distal end 3a that abuts the aerosol-generating portion 2.

The mouthpiece 3 illustrated in FIG. 1 is located at the mouth end of the article 1 and comprises three elements, a mouthpiece body 14 downstream of a cooling section 13, and a hollow tubular element 15 downstream of the mouthpiece body 14. In other embodiments, one or two of these different mouthpiece elements may be omitted or duplicated, and/or the elements may be provided in a different sequence. For example, the hollow tubular element 15 may be omitted, and the mouthpiece body 14 may form the mouth end of the article. In some embodiments where the hollow tubular element 15 is omitted, the length of the mouthpiece body 14 may be increased, or a further body of material may be provided at the mouth end.

In an alternative arrangement that is not illustrated, the mouthpiece comprises a mouthpiece body at the mouth end of the mouthpiece, optionally comprising a plug of cellulose acetate tow or paper, and optionally including capsule. Adjacent to this mouthpiece body is a first tube, optionally formed from paper, which abuts at its other end a further tubular section, optionally comprising cellulose acetate.

In the mouthpiece shown in FIG. 1, the cooling section 13, mouthpiece body 14 and hollow tubular element 15 are connected by a combining wrapping material 11.

As shown in FIG. 1, tipping paper 9 is wrapped around the full length of the mouthpiece 3 and over part of the aerosol generating portion 2. The tipping paper 9 has an adhesive on its inner surface (not shown) to connect the mouthpiece 3 and rod 2. In the illustrated embodiment, the tipping paper 9 extends about 5 mm over the rod of aerosol generating material 2 but it can alternatively extend from about 3 mm to about 15 mm over the rod 2, or from about 4 mm to about 6 mm, to provide a secure attachment between the mouthpiece 3 and rod 2.

In the illustrated embodiment, the article 1 is provided with first and second parallel rows of perforations 12 through the tipping material 9, combining wrapping material 11 and cooling section 13, providing ventilation into the mouthpiece 3 at the cooling section 13. The perforations 12 shown are formed as laser perforations, at positions about 18 mm and about 19 mm respectively from the downstream, mouth-end 3b of the mouthpiece 3. In other embodiments, the ventilation can be provided into the mouthpiece 3 at other locations.

Ambient air drawn into the mouthpiece 3 through the ventilation apertures 12 acts to cool and reduce the temperature of the mouthpiece 3. The ventilation area is configured such that when the mouthpiece 3 is placed between a user's lips, and an aerosol generated by the aerosol-generating composition 2 is drawn into the user's mouth through the cooling section 13 and mouthpiece 3, air is drawn into the cooling section 13 through the ventilation apertures 12 to cool the mouthpiece 3.

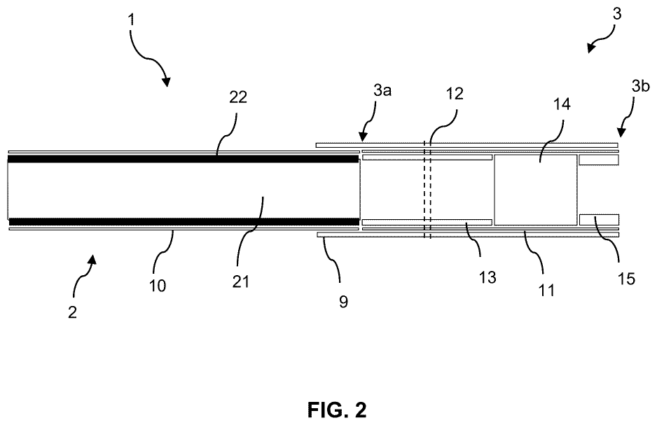

FIG. 2 shows an article 1 with a similar overall construction to the article shown in FIG. 1 and described above. Specifically, the mouthpiece and wrappers are the same.

In FIG. 2, the rod comprising the aerosol-generating composition is different to that shown in FIG. 1. In this embodiment, the aerosol-generating portion comprises a rod or segment comprising the first aerosol-generating material 21. This may be a rod of shredded botanical material. The entire length of this rod or segment is surrounded by a sheet 22 of a second aerosol-generating material comprising a thin film. In this embodiment, the two aerosol-generating materials are provided together but they are not blended and they are present in the aerosol-generating portion is different and distinct or separate forms. In the embodiment illustrated in FIG. 2 both of the two aerosol-generating materials are present along the entire length of the aerosol-generating portion 2.

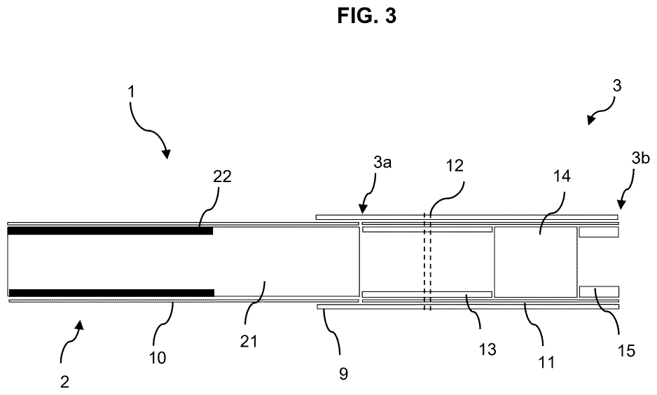

FIG. 3 shows an article 1 with a similar overall construction to the article shown in FIG. 2 and described above. Specifically, the mouthpiece and wrappers are the same.

In FIG. 3, the aerosol-generating portion 2 differs from that shown in FIG. 2 in that only part of the length of the rod or segment comprising the first aerosol-generating material 21 is surrounded by a sheet 22 of a second aerosol-generating material comprising a thin film. Once again, the first aerosol-generating material 21 may be shredded botanical material. Thus, in the embodiment illustrated in FIG. 3, only the first aerosol-generating material 21 is present along the entire length of the aerosol-generating portion 2. The second aerosol-generating material 22 is present over only part of the length of the aerosol-generating portion 2. This arrangement means that the second aerosol-generating material 22 may be heated and generate an aerosol at specific times during the overall use of the article.

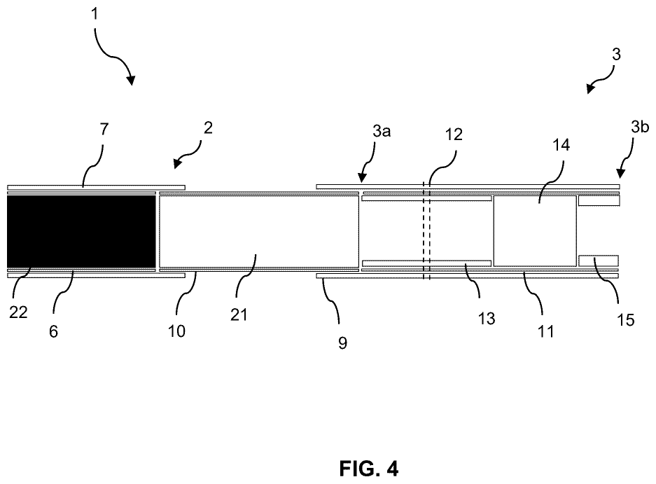

FIG. 4 shows an article 1 with a different construction of aerosol-generating portion 2. Once again, the mouthpiece and wrappers are the same as shown in the foregoing figures and are as described above.

In FIG. 4, the aerosol-generating portion 2 comprises two separate segments. The first segment is a rod or plug comprising the first aerosol-generating material 21. This segment abuts the distal end 3a of the mouthpiece 3. The second segment of the aerosol-generating portion 2 is a rod or plug comprising the second aerosol-generating material 22 comprising a thin film. This second segment is formed from a roll of a sheet of the second aerosol-generating material forming a plug. In alternative embodiments, the rod or plug may comprise a folded or gathered sheet of the second aerosol-generating material 22, aligned strips of the second aerosol-generating sheet material, or a plug of cut or shredded second aerosol-generating sheet material.

In the embodiment illustrated in FIG. 4, the first aerosol-generating material 21 and second aerosol-generating material 22 are present at different locations along the length of the aerosol-generating portion 2. This will allow them to be separately or independently heated, if desired.

In FIG. 4, the two segments of the aerosol-generating portion 2 are separately wrapped by wrappers 10 and 6. The two segments are held together by a tipping paper 7 which is wrapped around the full length of the segment comprising the second aerosol-generating material 22 and over part of the segment comprising the first aerosol generating material 21. The tipping paper 7 has an adhesive on its inner surface (not shown) to connect the segments of the aerosol-generating portion 2. In the illustrated embodiment, the tipping paper 7 extends about 5 mm over the segment comprising the first aerosol generating material 21 but it can alternatively extend over the entire length of the aerosol-generating portion 2, or even over the entire length of the article 1.

### Aerosol Provision System

In another aspect is provided an aerosol provision system for heating an aerosol generating composition to volatilize at least one component of the aerosol generating composition. The provision system comprises the aerosol generating article as disclosed herein and a heating device configured to receive at least a portion of the article and to heat the aerosol generating composition to volatilize at least one component of the aerosol generating composition. The heating device is configured to heat but not burn the aerosol generating composition (also referred to herein as the aerosolizable material).

In some embodiments, the heating device is configured to externally heat the aerosol generating composition, inductively heat the aerosol generating composition, resistively heat the aerosol generating composition, or a combination thereof. The heating device comprises a heater.

The heater may be, in some embodiments, a thin film, electrically resistive heater. In other embodiments, the heater may be an induction heater or the like. The heater may also be a combustible heat source or a chemical heat source which undergoes an exothermic reaction to product heat in use. The aerosol provision system may comprise a plurality of heaters. The heater(s) may be powered by a battery.

In some embodiments, the heater may heat, without burning, the aerosolizable material to a temperature of from about 120° C. to about 350° C. in use. In some embodiments, the heater may heat, without burning, the aerosolizable material to a temperature of from about 140° C. to about 250° C. in use. In some embodiments in use, substantially all of the thin film is no more than about 4 mm, about 3 mm, about 2 mm or about 1 mm from the heater. In some embodiments, the thin film is disposed from about 0.01 mm to about 2 mm from the heater, suitably from about 0.02 mm to about 1 mm, suitably from about 0.1 mm to about 0.5 mm. These minimum distances may, in some embodiments, reflect the thickness of a carrier that supports the thin film. In some embodiments, a surface of the thin film and/or of the second aerosol-generating material may directly abut the heater.

In some embodiments, the heater may be embedded in the aerosol generating composition. In some such embodiments, the heater may be an electrically resistive heater (with exposed contacts for connection to an electrical circuit). In other such embodiments, the heater may be a susceptor embedded in the aerosol-generating composition, which is heated by induction.

In some embodiments, the aerosol provision system may be a heat-not-burn device. A heat-not-burn device is disclosed in WO 2015/062983 A2, which is incorporated by reference in its entirety.

In some embodiments, the aerosol provision system may be an electronic tobacco hybrid device. That is, it may contain a solid aerosol generating composition and a liquid aerosolizable material. The separate aerosolizable materials may be heated by separate heaters, the same heater or, in one case, a downstream solid aerosol-generating composition may be heated by a hot aerosol which is generated from the upstream aerosolizable material. An electronic hybrid device is disclosed in WO 2016/135331 A1, which is incorporated by reference in its entirety.

The aerosol provision system may comprise an integrated aerosol generating article and heater, or may comprise a heater device into which the article is inserted in use. In either case, as described above, the heater is configured to heat but not burn the aerosol generating composition.

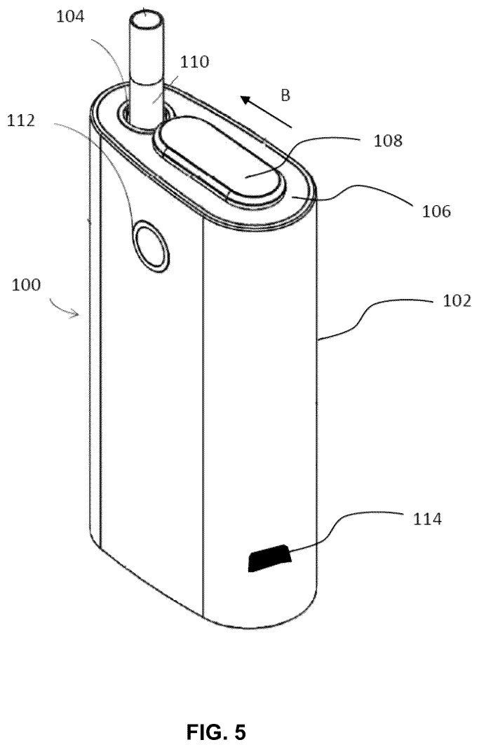

FIG. 5 shows an example of a non-combustible aerosol provision device 100 for generating aerosol from an aerosol-generating composition of an article or consumable 110, as described herein. For example, the article 110 may be any one of the articles 1 shown in FIGS. 1 to 4.

In broad outline, the device 100 may be used to heat a replaceable article 110 comprising the aerosol-generating composition as described herein, for instance an article as described elsewhere herein, to generate an aerosol or other inhalable medium which is inhaled by a user of the device 100. The device 100 and replaceable article 110 together form a system.

The device 100 comprises a housing 102 (in the form of an outer cover) which surrounds and houses various components of the device 100. The device 100 has an opening 104 in one end, through which the article 110 may be inserted for heating by a heating assembly. In use, the article 110 may be fully or partially inserted into the heating assembly where it may be heated by one or more components of the heater assembly. The article 110 is illustrated having a rod-shape, like the articles 1 illustrated in FIGS. 1 to 4.

The device 100 of this example comprises a first end member 106 which comprises a lid 108 which is moveable relative to the first end member 106 to close the opening 104 when no article 110 is in place. In FIG. 5, the lid 108 is shown in an open configuration, however the lid 108 may move into a closed configuration. For example, a user may cause the lid 108 to slide in the direction of arrow “B”.

The device 100 may also include a user-operable control element 112, such as a button or switch, which operates the device 100 when pressed. For example, a user may turn on the device 100 by operating the switch 112. This switch may also actuate the means for opening the container inserted into the device, in readiness for its use.

The device 100 may also comprise an electrical component, such as a socket/port 114, which can receive a cable to charge a battery of the device 100. For example, the socket 114 may be a charging port, such as a USB charging port.

### Method of Manufacture

The disclosure also provides a method of making an aerosol generating article as described herein. Specifically, the method comprises the steps of:
providing an aerosol generating composition, a mouth end section and a wrapping material;
combining the aerosol generating portion and mouthpiece with the wrapping material to form a component; and
providing ventilation into the article.

In some embodiments, the ventilation is provided into the mouthpiece of the article.

In some embodiments, the provision of ventilation into the article comprises forming holes or apertures to allow ambient air to be drawn into the article. In some embodiments the apertures are formed using a laser. In some embodiments, the apertures allow air to be drawn into a cooling section, a filter plug and/or a mouth end hollow tube element of the article.

In other embodiments, the provision of ventilation into the article comprises forming a porous section of the side wall of the article by surrounding the article with a porous wrapper. In some embodiments, the porous wrapper is positioned to allow air to be drawn into a cooling section, a filter plug and/or a mouth end hollow tube element of the article.

The method may comprise making the aerosol generating composition as described herein and incorporating it into an aerosol generating article.

The method may comprise (a) forming a slurry comprising components of the thin film or precursors thereof, (b) forming a layer of the slurry, (c) optionally setting the slurry to form a gel, and (d) drying to form a thin film. In some embodiments, the slurry comprises the active agent (e.g., a substituted 3-(1-methylpyrrolidin-2-yl)pyridine as described herein). In some embodiments, the slurry does not include the active agent, and the active agent is added as a “top dressing” to the film before or after drying as described herein below.

The step (b) of forming a layer of the slurry may comprise spraying, casting or extruding the slurry, for example. In some embodiments, the layer is formed by electrospraying the slurry. In some embodiments, the layer is formed by casting the slurry.

In some embodiments, the slurry is applied to a carrier.

In some embodiments, the steps (b) and/or (c) and/or (d) may, at least partially, occur simultaneously (for example, during electrospraying). In some embodiments, these steps may occur sequentially.

In some embodiments, the slurry has a viscosity of from about 10 to about 20 Pa-s at 46.5° C., such as from about 14 to about 16 Pa-s at 46.5° C. In some embodiments, the slurry may have an elastic modulus of from about 5 to about 1200 Pa (also referred to as storage modulus); in some embodiments, the slurry may have a viscous modulus of from about 5 to about 600 Pa (also referred to as loss modulus).

The step (c) of setting the gel may comprise the addition of a setting agent to the slurry. For example, the slurry may comprise sodium, potassium or ammonium alginate as a gelling agent, and a setting agent comprising a calcium source (such as calcium chloride), may be added to the slurry to form a calcium alginate gel.

The total amount of the setting agent, such as a calcium source, may be from about 0.5 to about 5 wt % (calculated on a dry weight basis). The addition of too little setting agent may result in a thin film which does not stabilize the thin film components and results in these components dropping out of the thin film. The addition of too much setting agent results in a thin film that is very tacky and/or too brittle, and consequently has poor handleability.

In some embodiments however, no setting agent is needed because the botanical extract contains sufficient calcium to effect gelation. This may, for example, be true of tobacco extracts.

Alginate salts are derivatives of alginic acid and are typically high molecular weight polymers (10-600 kDa). Alginic acid is a copolymer of β-D-mannuronic (M) and α-L-guluronic acid (G) units (blocks) linked together with (1,4)-glycosidic bonds to form a polysaccharide. On addition of calcium cations, the alginate crosslinks to form a gel.

In some embodiments, a binder is used that does not require a setting agent, such as a cellulose ether (e.g., hydroxypropyl cellulose or carboxymethyl cellulose).

In some embodiments, the slurry solvent may consist essentially of, or consist of, water. In some embodiments, the slurry may comprise from about 50 wt %, about 60 wt %, about 70 wt %, about 80 wt % or about 90 wt % of solvent (WWB).

In embodiments where the solvent consists of water, the dry weight content of the slurry will match the dry weight content of the thin film. Thus, the discussion herein relating to the solid composition is explicitly disclosed in combination with the slurry aspect of the disclosure.

In some embodiments, the active agent (e.g., a substituted 3-(1-methylpyrrolidin-2-yl)pyridine as described herein) is added to the thin film. For example, in some embodiments, at least a portion or even all of the active agent may be applied during or after drying (e.g., by spraying it on or wiping it onto the sheet). The substituted 3-(1-methylpyrrolidin-2-yl)pyridine is generally applied as a solution in, for example, an aerosol former such as glycerol, propylene glycol, or a mixture thereof. Without wishing to be bound by theory, it is believed that adding the substituted 3-(1-methylpyrrolidin-2-yl)pyridine solely after film formation, either during or after drying, may reduce the potential for contamination and/or increase the homogeneity of distribution of the substituted 3-(1-methylpyrrolidin-2-yl)pyridine in the film of aerosol generating material.

Many modifications and other implementations of the disclosure will come to mind to one skilled in the art to which this disclosure pertains having the benefit of the teachings presented in the foregoing descriptions and the associated figures. Therefore, it is to be understood that the disclosure is not to be limited to the specific implementations disclosed herein and that modifications and other implementations are intended to be included within the scope of the appended claims. Although specific terms are employed herein, they are used in a generic and descriptive sense only and not for purposes of limitation.

### EXAMPLES

### Example 1. Preparation of an Aerosol Generating Composition—Top Dressing

An aerosol generating composition according to an embodiment of the disclosure is prepared. A tobacco extract is obtained by extraction of tobacco with deionized and purified water. The extract comprises 3.1 wt % nicotine and has a solids content (including nicotine) of 62.9 wt % (wet weight basis). A premix of glycerol and guar gum was formed and set aside. Wood pulp and water (97% moisture) are added to a 10-litre glass or metal beaker and mixed with a Silverson L5M-A Laboratory Mixer at a speed of 1200 rpm for 2 minutes. Ground cellulose (ARBOCEL B 600) is slowly added over a period of 2 minutes into the wood pulp mixture, and the speed increased to 2000 rpm for an additional 2 minutes. The tobacco extract is slowly added over 2 minutes into the slurry mix, keeping the mixer speed at 2000 rpm for a further 3 minutes. The premix of glycerol and guar gum is slowly added to the slurry over 2 minutes at a speed of 3000 rpm and blended for an additional 5 minutes. The resulting slurry has the composition provided in Table 2.

TABLE 2
Slurry composition

| Component | wt % (WWB) |
|---|---|
| Tobacco Extract | 45% |
| Wood pulp | 11% |
| Guar gum | 4% |
| Glycerol | 18% |
| Ground cellulose | 22% |

A casting knife is set between 0.10 to 0.13 cm and the slurry cast into sheets on a metal plate. The sheets are dried at a temperature between 70° C. and 120° C. for 3 hours in an oven.

In an alternative casting process, the slurry is cast onto a metal plate to a thickness of 2 mm and allowed to set to form a gel. Once the gel has set, it is dried in an oven at 65° C. for approximately 2 hours to provide a thin film having approximately 10 wt % water and a thickness of about 0.2 mm.

To the thin film is added the substituted 3-(1-methylpyrrolidin-2-yl)pyridine by spraying it onto the thin film as a solution in a suitable solvent. The spraying onto the film may be performed before, during, or after any further processing, such as shredding the film.

### Example 2. Preparation of an Aerosol Generating Composition

An aerosol generating composition according to an embodiment of the disclosure is prepared as in Example 1, except that the thin film is shredded and combined with a botanical material as described herein (e.g., rooibos). To the mixture of shredded film and botanical is added the substituted 3-(1-methylpyrrolidin-2-yl)pyridine by spraying a solution on the mixture, such as a solution of the substituted 3-(1-methylpyrrolidin-2-yl)pyridine in an aerosol former (e.g., glycerin, propylene glycol, or a mixture thereof).

### Example 3. Preparation of an Aerosol Generating Composition—Active in Slurry

An aerosol generating composition according to an embodiment of the disclosure is prepared as in Example 1, except that the substituted 3-(1-methylpyrrolidin-2-yl)pyridine is added to the slurry prior to casting. Adding the substituted 3-(1-methylpyrrolidin-2-yl)pyridine prior to setting of the binder and drying ensures good homogeneity of the composition.

### Example 4. Evaluation of Active Agent Concentration in Aerosol

The transfer efficiency to aerosol of 2-methyl-5-(1-methylpyrrolidin-2-yl)pyridine from a substrate impregnated with 2-methyl-5-(1-methylpyrrolidin-2-yl)pyridine was evaluated and compared against the transfer efficiency of nicotine to aerosol from a control substrate impregnated with nicotine.

An aerosol generating composition in the form of a stick comprising a shredded thin film comprising cellulose, glycerol, wood pulp, and binder was impregnated with 2-methyl-5-(1-methylpyrrolidin-2-yl)pyridine. The impregnation was performed by injecting a solution of 12% 2-methyl-5-(1-methylpyrrolidin-2-yl)pyridine in glycerin diluted with 95% ethanol into the shredded film of the stick. Two versions were formed, one injected with 0.56 mg of 2-methyl-5-(1-methylpyrrolidin-2-yl)pyridine (denoted “Low 6MP”) and one injected with 1.12 mg of 2-methyl-5-(1-methylpyrrolidin-2-yl)pyridine (denoted “High 6MP”). As the control, sticks having the same base composition were injected with a solution of 12% nicotine in glycerin diluted with 95% ethanol to form sticks with 1.12 mg of added nicotine.

Aerosolization testing was conducted using two aerosol delivery devices: a GLO™ Hyper Pro device and a GLO™ Hyper Air device. The testing was conducted using a 55/2/30 puff regime at a heating temperature of 285° C. for the GLO™ Hyper Pro device and a heating temperature of 265° C. for the GLO™ Hyper Air device. The amount of active (2-methyl-5-(1-methylpyrrolidin-2-yl)pyridine or nicotine) was determined by quantitative liquid chromatography using a UPLC instrument with a PDA detector. The % transfer was then calculated by dividing the amount of active in the aerosol by the total amount of active present.

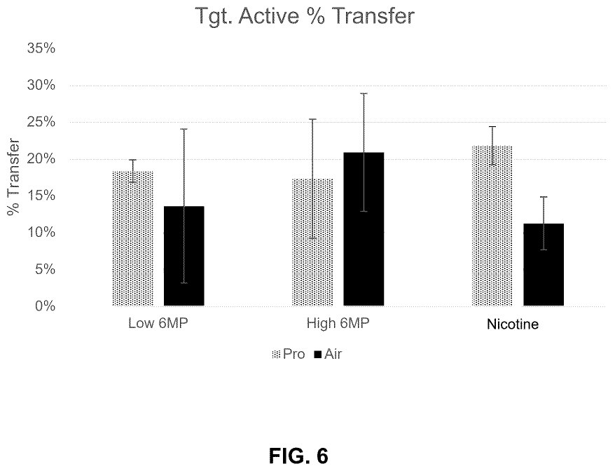

FIG. 6 is a graphical depiction showing the transfer efficiency of 2-methyl-5-(1-methylpyrrolidin-2-yl)pyridine from an aerosol generating rod to aerosol for embodiments of the disclosure as compared to a that of control aerosol generating rod including nicotine. With reference to FIG. 6, the transfer efficiency from substrate to aerosol for 2-methyl-5-(1-methylpyrrolidin-2-yl)pyridine from the two devices at two different concentrations and two different heating temperatures was between about 12 and about 20%, which is comparable to the transfer efficiency of nicotine under the same conditions.

1. An aerosol generating composition comprising a thin film, the thin film comprising a binder, an aerosol former, and an active agent, the active agent comprising at least a compound having a structure according to Formula I, Formula II, or Formula III: 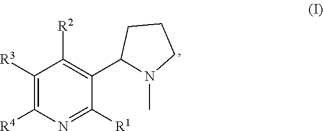
wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or
the substituted 3-(1-methylpyrrolidin-2-yl)pyridine has a structure according to Formula II: 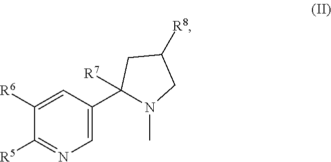
wherein:
R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C₁-C₃ alkyl; and
at least one of R⁷ and R⁸ is not hydrogen; or 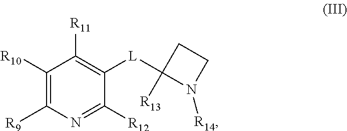
wherein
L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

2. The aerosol generating composition of claim 1, wherein the active agent comprises a compound having a structure according to Formula I, and wherein R¹, R², and R³ are each H.

3. The aerosol generating composition of claim 2, wherein R⁴ is optionally substituted C₁-C₆ alkyl, F, Cl, Br, OCH₃, OCH₂CH₃, or CN.

4. The aerosol generating composition of claim 3, wherein R⁴ is C₁-C₃ alkyl, such as wherein R⁴ is CH₃.

5. The aerosol generating composition of claim 1, wherein the active agent comprises a compound having a structure according to Formula II, and wherein R⁵ and R⁶ are H; R⁷ is CH₃; and R⁸ is H.

6. The aerosol generating composition of claim 1, wherein the active agent comprises a compound having a structure according to Formula II, and wherein R⁵, R⁶ and R⁷ are H, and R⁸ is CH₃.

7. The aerosol generating composition of claim 1, wherein the thin film further comprises a botanical extract, a tobacco extract, or a combination thereof.

8. The aerosol generating composition of claim 1, further comprising a botanical material.

9. The aerosol generating composition of claim 8, wherein the botanical material comprises tobacco.

10. The aerosol generating composition of claim 8, wherein the botanical material comprises a botanical extract.

11. The aerosol generating composition of claim 8, wherein the botanical material comprises a shredded non-tobacco botanical material; optionally, wherein the shredded non-tobacco botanical material comprises or is rooibos.

12. The aerosol generating composition of claim 8, comprising from about 50 to about 95% of the botanical material and from about 5 wt % to about 50 wt % of the thin film.

13. The aerosol generating composition of claim 8, wherein the thin film is present as a cut or shredded sheet which is blended with the botanical material.

14. The aerosol generating composition of claim 8, comprising a plug or section comprising the botanical material, wherein the thin film is present in the form of a sheet circumscribing at least a portion of the plug or section.

15. The aerosol generating composition of claim 8, further comprising a paper wrapper circumscribing the aerosol generating composition, and wherein the thin film is present in the form of a sheet positioned between the botanical material and the wrapper.

16. The aerosol generating composition of claim 8, comprising a first section comprising the botanical material and a second section comprising the thin film in the form of a rolled sheet.

17. The aerosol generating composition of claim 1, wherein the active agent further comprises a nicotine component, a cannabinoid, a terpene, caffeine, an amino acid, a vitamin, melatonin, a botanical extract, or a combination thereof.

18. The aerosol generating composition of claim 1, wherein the active agent further comprises a nicotine component.

19. The aerosol generating composition of claim 1, wherein the aerosol generating composition is substantially free of a nicotine component.

20. The aerosol generating composition of claim 1, further comprising a second thin film, the second thin film comprising a binder, an aerosol former, and optionally, an active agent.

21. An aerosol generating article configured for use in an aerosol provision system, the aerosol generating article comprising the aerosol generating composition of claim 1.

22. An aerosol provision system for heating an aerosol generating composition to volatilize at least one component of the aerosol generating composition, the system comprising:
the aerosol generating article of claim 21; and
a heating device configured to receive at least a portion of the article and to heat the aerosol generating composition to volatilize at least one component of the aerosol generating composition.

23. The aerosol provision system of claim 22, wherein the heating device is configured to externally heat the aerosol generating composition, inductively heat the aerosol generating composition, resistively heat the aerosol generating composition, or a combination thereof.
